(19) United States
(12) Patent Application Publication
(10) Pub. No.: US 2026/0160760 A1
(43) Pub. Date: Jun. 11, 2026

(54) DETECTION OF BINDING PROTEINS IN EQUILIBRIUM AND NON-EQUILIBRIUM CONDITIONS

(71) Applicant: Nautilus Subsidiary, Inc., Seattle, WA (US)

(72) Inventor: Gregory KAPP, San Carlos, CA (US)

(21) Appl. No.: 19/407,511

(22) Filed: Dec. 3, 2025

Related U.S. Application Data
(60) Provisional application No. 63/729,089, filed on Dec. 6, 2024.

(51) Int. Cl. G01N 33/543; C12Q 1/25; C12Q 1/6806; C12Q 1/686; G01N 33/68

(52) U.S. Cl. CPC G01N 33/54313; C12Q 1/25; C12Q 1/6806; C12Q 1/686; G01N 33/6845; G01N 2333/9015

(57) ABSTRACT

Methods, systems, and compositions are provided for characterizing the interactions of binding reagents with ligand-binding proteins, such as enzymes. Unknown ligand-binding proteins may be identified at single-molecule resolution based upon their interactions with binding reagents. Proteins with unknown ligand-binding behaviors may be characterized by binding reagent methods set forth herein.

### CROSS-REFERENCE TO RELATED APPLICATIONS

This application claims priority to U.S. Provisional Application No. 63/729,089, filed on Dec. 6, 2024, which is incorporated herein by reference in its entirety.

### BACKGROUND

Recognition and binding of substrates and/or cofactors is a common attribute of enzymes and other ligand-binding proteins. An enzyme or other ligand-binding protein will typically contain at least one, and often multiple, binding sites that are configured to bind a substrate or cofactor. For certain proteins, binding of a substrate or cofactor to a binding site of the protein may trigger conformational changes in the protein, thereby exposing or hiding another binding site. The arrangement of amino acid residues in the protein's secondary and tertiary structures forms a binding site and confers binding specificity to the binding site. The binding specificity of a protein binding site may be high enough that the protein will bind a substrate but not bind a structurally similar derivative of the substrate. However, in some cases, a binding site of a protein may bind a molecule that is structurally dissimilar from the substrate. A binding site may bind to only a portion of a substrate or cofactor, while the remainder of the molecule exerts a lesser influence on the binding of the molecule to the protein.

As an example, tRNA synthetases are a family of enzymes that catalyze the attachment of amino acids to transfer RNAs during the protein translation process. tRNA synthetases are multi-domain enzymes that facilitate a multi-step process when attaching amino acids to transfer RNAs, including catalyzing the activation of amino acids by reacting the amino acids with molecules of ATP, and catalyzing the attachment of the activated amino acids to the transfer RNAs. tRNA synthetases may further include an editing domain that can recognize mispaired amino acid-tRNA complexes and cleave the amino acids from the complexes.

A tRNA synthetase molecule must be capable of recognizing and specifically binding to several molecules, including ATP, at least one amino acid, and at least one tRNA. The binding specificity of a tRNA synthetase for an amino acid is determined in part by non-covalent interactions of the sidechain of the bound amino acid molecule with the amino acid residues that form the amino acid binding site of the tRNA synthetase. The portion of the amino acid containing the amine and carboxyl functional groups has less influence on the binding of an amino acid to a tRNA synthetase. Although most organisms have a complete set of tRNA synthetases for the proteinogenic amino acids, the structure of the molecular binding sites of the tRNA synthetases can vary from organism to organism. Moreover, tRNA synthetases may play a role in some disease states.

### SUMMARY

In an aspect, provided herein is a method, comprising: (a) providing a plurality of polypeptides, wherein each polypeptide of the plurality of polypeptides is individually co-localized with a first unique identifier, and wherein each polypeptide of the plurality of polypeptides has an unknown identity, (b) contacting to the plurality of polypeptides a plurality of binding reagents, wherein each binding reagent comprises a protein substrate or enzymatic cofactor, and wherein the protein substrate or enzymatic cofactor is attached to a second unique identifier, (c) detecting for each polypeptide of the plurality of polypeptides presence or absence of a co-localized signal from the second unique identifier with the first unique identifier of the polypeptide, and (d) for each polypeptide with the co-localized signal from the second unique identifier with the first unique identifier, assigning protein identity to the polypeptide.

In another aspect, provided herein is a method, comprising: (a) providing a library of binding reagents, wherein each binding reagent of the library of binding reagents comprises a protein substrate or enzymatic cofactor attached to a first unique identifier, wherein the library of binding reagents comprises a plurality of structurally-unique binding reagents, each structurally unique binding reagent comprising a differing attachment conformation of the protein substrate or enzymatic cofactor to the first unique identifier, and each structurally unique binding reagent comprising a first unique identifier that differs from the first unique identifier of any other structurally-unique binding reagent, (b) contacting a protein molecule with the library of binding reagents; wherein the protein molecule is co-localized with a second unique identifier, and (c) detecting co-localization of a first unique identifier of a structurally unique binding reagent with the second unique identifier, thereby identifying a portion of the protein substrate or enzymatic cofactor that is bound by the protein molecule.

In another aspect, provided herein is a method, comprising: (a) providing a library of binding reagents, wherein each binding reagent of the library of binding reagents comprises a protein binding candidate attached to a first unique identifier, wherein the library of binding reagents comprises a plurality of structurally-unique binding reagents, each structurally unique binding reagent comprising a differing enzyme binding candidate, and each structurally unique binding reagent comprising a first unique identifier that differs from the first unique identifier of any other structurally-unique binding reagent, (b) contacting a protein molecule with the library of binding reagents; wherein the protein molecule is co-localized with a second unique identifier, and (c) detecting co-localization of a first unique identifier of a structurally unique binding reagent with the second unique identifier, thereby identifying a molecule that binds to the protein molecule.

In another aspect, provided herein is a method, comprising: (a) contacting a protein molecule to a binding reagent in the presence of a molecular activator, wherein the protein molecule is co-localized with a first unique identifier, wherein the binding reagent comprises a protein substrate, and wherein the protein substrate is attached to a second unique identifier, and (b) detecting a co-localized signal from the second unique identifier of the binding reagent and the first unique identifier of the polypeptide, thereby identifying binding of the protein substrate to the protein molecule.

In another aspect, provided herein is a method, comprising: (a) providing a plurality of polypeptides, wherein the plurality of polypeptides is immobilized on a plurality of sites of a solid support, wherein each site of the plurality of sites is attached to only one polypeptide of the plurality of polypeptides, and wherein each site of the plurality of sites is optically resolvable from each other site of the plurality of sites, (b) contacting to the solid support a plurality of binding reagents, wherein each binding reagent comprises a protein substrate or enzymatic cofactor, and wherein the protein substrate or enzymatic cofactor is attached to a detectable label, (c) detecting for each site of the plurality of sites a presence or absence of a signal from a detectable label, and (d) for each site of the plurality of sites having a detected presence of a signal from the detectable label, assigning a protein identity to the polypeptide of the plurality of polypeptides attached to the site.

In another aspect, provided herein is a method, comprising: (a) combining in a fluid phase a plurality of polypeptides with a plurality of binding reagents, wherein each polypeptide of the plurality of polypeptides is individually attached to a first unique identifier, wherein each polypeptide of the plurality of polypeptides has an unknown identity, wherein each binding reagent comprises a protein substrate or enzymatic cofactor, and wherein the protein substrate or enzymatic cofactor of each individual binding reagent is attached to a second unique identifier, (b) coupling binding reagents of the plurality of binding reagents to polypeptides of the plurality of polypeptides, (c) for each polypeptide of the polypeptides bound to a binding reagent of the plurality of binding reagents, forming an interaction identification moiety, wherein the interaction identification comprises the first unique identifier of the polypeptide and the second unique identifier of the binding reagent, (d) detecting each formed interaction identification moiety, and (e) for each detected interaction identification moiety, determining an identity of the polypeptide from which the interaction identification moiety was obtained.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A, 1B, 1C, 1D, 1E, 1F, and 1G illustrate configurations of a system for optically detecting binding interactions between protein substrates or enzymatic cofactors and an enzyme molecule, in accordance with some embodiments.

FIGS. 2A, 2B, 2C, 2D, 2E, 2F, 2G, 2H, and 2I depict configurations of a system for non-optically detecting binding interactions between protein substrates or enzymatic cofactors and an enzyme molecule, in accordance with some embodiments.

FIGS. 3A, 3B, 3C, and 3D display configurations of a system for forming an interaction identification moiety, in accordance with some embodiments.

FIG. 4 shows various members of a library of binding reagents, in which each member has a unique configuration of attachment of a protein substrate or enzymatic cofactor to a linking moiety, or in which each member is a derivative of the protein substrate or enzymatic cofactor, in accordance with some embodiments.

FIG. 5 is a schematic drawing of a system for performing embodiments of the techniques, reagents, systems, and methods described herein.

### DETAILED DESCRIPTION

Enzymes are a broad class of proteins that catalyze chemical reactions in biological systems. Many enzymes function within a cell or a similar biological structure, for example, within metabolic and degradative pathways. In some cases, enzymes are secreted by a cell into an extracellular environment to adapt or regulate the environment surrounding the cell. The roles of enzymes in biological systems are ubiquitous, and a large number of cellular or biological processes are mediated by the action of an enzyme.

An enzyme will typically act upon a substrate to alter the substrate into a product. The ability of the enzyme to act upon the substrate is dependent upon binding of the substrate to the enzyme at a binding or active site of the enzyme. In some cases, an enzyme will catalyze a reaction involving two substrate molecules such as an addition of the two molecules together, or a transfer of a moiety from one molecule to the other molecule. In such cases, an enzyme may contain at least two binding sites, each binding site having a binding specificity for one of the enzymatic substrates. In some cases, an enzyme will utilize an enzymatic cofactor to catalyze a reaction. An enzymatic cofactor may comprise an organic or inorganic moiety that is incorporated into the enzyme to facilitate the catalytic function of the enzyme. Enzymatic cofactors can facilitate processes such as electron transfer and transfer of functional groups such as protons and methyl groups. An enzymatic cofactor may be incorporated into an enzyme during folding of the enzyme during or after translation, or may be incorporated in a transient fashion (i.e., associating and dissociating from the enzyme throughout the enzyme's function).

The binding of a substrate to a binding site of an enzyme can be highly specific to the structure of the substrate. Minor changes in the structure of the substrate, such as a change in chirality or a minor substitution (e.g., a methyl group in place of a proton) can inhibit, or altogether eliminate, the ability of the substrate to bind to the binding site of an enzyme. However, the binding site of an enzyme may not bind to the entirety of a substrate molecule, and changes to non-binding portions of the substrate molecule may not affect the ability of the substrate molecule to bind to the binding site of the enzyme. This may be true of many types of enzymes, including tRNA synthetases and enzymes that perform post-translational modifications of diverse proteins.

tRNA synthetases are a family of enzymes that catalyze the attachment of amino acids to transfer RNA molecules during the protein translation process. Each tRNA synthetase molecule can specifically bind a particular amino acid within an amino acid binding site and can further bind a larger tRNA molecule to a portion of the enzyme adjacent to the amino acid binding site. The highly-specific binding of an amino acid to an active site of a tRNA synthetase may be driven primarily by the structure of the sidechain of the amino acid, as the portion of the amino acid that forms the peptide chain is invariant across all amino acids. Further, the tRNA synthetase molecule can catalyze the activation of the amino acid by binding a molecule of ATP and then binding it to the amino acid, thereby releasing a molecule of pyrophosphate. The activated amino acid-AMP molecule can then be bound to the tRNA, thereby releasing the AMP molecule. tRNA synthetases often further comprise an editing binding site that can recognize incorrectly paired amino acid-tRNA complexes and catalyze the release of the amino acid from the tRNA. Accordingly, each unique tRNA synthetase may comprise multiple binding sites, each having a binding specificity for a different molecule.

The methods and systems of the present disclosure can be extended to non-enzymatic proteins that are characterized by binding. Binding of either small molecules (e.g., molecules having a molecular weight of less than 1 kiloDalton) or macromolecules (e.g., molecules having a molecular weight of greater than or equal to 1 kiloDalton) may occur for a variety of non-enzymatic proteins in cellular processes such as formation of structural or other protein complexes, intra- or extra-cellular transport, signaling, and regulation of enzyme-mediated processes (e.g., transcriptional up-regulation or down-regulation). Like the structural and/or chemical specificity of enzyme binding, non-enzymatic proteins can have binding sites that form binding interactions to substrates or cofactors with high specificity. Accordingly, methods and compositions set forth herein may be extended to include substrates and/or cofactors for non-enzymatic proteins.

The present disclosure provides methods and systems for identifying the presence of one or more enzymes or other ligand-binding proteins from within a population of unknown proteins. The methods and system may utilize probes comprising protein substrates or enzymatic cofactors to identify the presence of a protein in the population of unknown proteins. The present disclosure also provides methods and systems for characterizing proteins based on the binding of probes to the proteins. The methods and systems provided may be useful for identifying molecules (e.g., pharmaceutical compounds) that form binding interactions with an enzyme or other ligand-binding molecule.

### Definitions

Terms used herein will be understood to take on their ordinary meaning in the relevant art unless specified otherwise. Several terms used herein and their meanings are set forth below.

In some of the implementations described herein, the terms “active site” and “binding site,” when used in reference to a protein molecule, can refer synonymously to a region of the protein molecule that transiently contains a ligand, such as a substrate molecule and/or a cofactor molecule. An enzymatic binding site may further possess a catalytic activity involving a ligand bound to the binding site. A protein binding site may form a non-covalent interaction or a transient covalent interaction with a bound ligand. A protein molecule can possess a single binding site, or a plurality of binding sites. Each binding site of a protein molecule containing more than one binding site may possess a unique catalytic activity or a binding specificity for a differing ligand. A binding site may be configured to bind a plurality of structurally unique molecules.

In some of the implementations described herein, the terms “address” and “site” can refer synonymously to a location in an array where a particular analyte (e.g. protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses

In some of the implementations described herein, the term “affinity reagent” can refer to a molecule or other substance that is capable of specifically or reproducibly binding to an analyte (e.g. protein) or moiety (e.g. post-translational modification of a protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)₂ fragments, single-chain variable fragments (scFv), di-scFv, tri-scFv, or microantibodies), aptamers, affibodies, affilins, affimers, affitins, alphabodies, anticalins, avimers, miniproteins, DARPins, monobodies, nanoCLAMPs, lectins, or functional fragments thereof. The term “affinity agent” is intended to be synonymous with the term “affinity reagent.”

In some of the implementations described herein, the term “antibody” can refer to a protein that binds to an antigen or epitope via at least one complementarity determining region (CDR). An antibody can include all elements of a full-length antibody. However, an antibody need not be full length and functional fragments can be particularly useful for many uses. The term “antibody” as used herein encompasses full length antibodies and functional fragments thereof.

In some of the implementations described herein, the term “array” can refer to a population of analytes (e.g. proteins) that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

In some of the implementations described herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some of the implementations described herein, the term “binding affinity” or “affinity” can refer synonymously to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (KD), equilibrium association constant (KA), association rate constant (kₒₙ), dissociation rate constant (k_{off}) and the like. See, for example, Segel, Enzyme Kinetics John Wiley and Sons, New York (1975), which is incorporated herein by reference in its entirety.

In some of the implementations described herein, the term “binding interaction” can refer to a reaction that associates an affinity reagent to an analyte. A binding reaction may be a covalent or non-covalent interaction. A binding interaction may associate an affinity reagent to an analyte for a sufficient length of time to detect a complex formed by the affinity reagent and analyte.

In some of the implementations described herein, the term “binding reagent” can refer to a molecule, particle, or moiety that can form a binding interaction with another molecule as at least part of its activity or function. A binding reagent may form a covalent interaction or a non-covalent interaction with a molecule. A binding reagent may covalently modify a molecule to which the binding reagent binds. A binding reagent may comprise an affinity agent, as set forth herein. A binding reagent may comprise two or more affinity agents. A binding reagent may comprise two or more detectable labels. A binding reagent may comprise a particle (e.g., a nucleic acid nanoparticle, a polymer nanoparticle, a branched or dendrimeric nanoparticle) that couples one or more affinity agents to one or more detectable labels. Examples of multivalent binding reagents (i.e., binding reagents comprising two or more moieties configured to form binding interactions) are described in U.S. Pat. No. 11,692,217 and U.S. Patent Application No. 20230090454, each of which is herein incorporated by reference in its entirety.

In some of the implementations described herein, the term “binding specificity” refers to the tendency of a binding reagent to preferentially interact with a given analyte relative to other analytes. A binding reagent may have a calculated, observed, known, or predicted binding specificity for a given analyte. Binding specificity may refer to selectivity for a single analyte in a given sample relative to one, some or all other analytes in the sample. Moreover, binding specificity may refer to selectivity for a subset of analytes in a given sample relative to at least one other analyte in the sample.

In some of the implementations described herein, the term “co-localized” can refer to two or more entities with a minimally varying spatial and/or temporal proximity. Two entities may be co-localized if they are both immobilized at a same address of a solid support. Two entities may be co-localized if they are both attached to a same linking moiety. Two entities may be co-localized if their respective mass transfer through a fluid phase is not independent of each other. For example, two unbound entities in a fluid phase may be co-localized if they are joined by a linking moiety, thereby coupling their diffusions through the fluid phase over a long enough time period.

The term “comprising” is intended herein to be open-ended, including not only the recited elements, but further encompassing any additional elements.

In some of the implementations described herein, the term “each,” when used in reference to a collection of items, is intended to identify an individual item in the collection but does not necessarily refer to every item in the collection. Exceptions can occur if explicit disclosure or context clearly dictates otherwise.

In some of the implementations described herein, the term “enzymatic cofactor” can refer to a molecule or ligand that facilitates the catalytic function of an active site of an enzyme molecule. An enzymatic cofactor may be co-localized at an enzymatic active site with a protein substrate. Proper spatial conformation of an enzymatic active site may be dependent upon incorporation of an enzymatic cofactor into the secondary and/or tertiary structure of an enzyme molecule. An enzyme molecule may be transiently modified during the catalytic activity of an enzyme molecule, such as oxidation or reduction of the enzymatic cofactor, or modification of the enzymatic cofactor with a labile functional group, such as methylation, amination, carboxylation, etc. An enzymatic cofactor may be an organic molecule, an inorganic molecule, or a combination thereof.

In some of the implementations described herein, the term “epitope” can refer to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

In some of the implementations described herein, the term “fluid phase,” when used in reference to a molecule, can mean that the molecule is in a state wherein it is mobile in a fluid, for example, being capable of diffusing through the fluid.

In some of the implementations described herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

In some of the implementations described herein, the term “interaction identification moiety” can refer to a detectable moiety that is formed during the recording of a binding interaction. An interaction identification moiety can comprise an analyte-specific code or residue sequence that identifies the analyte that participated in the recorded binding reaction. An interaction identification moiety can further comprise one or more codes or residue sequences (e.g., a binding-reagent specific code, an assay sequence-specific code, a vessel-specific code) that provides information regarding the binding reagent that participated in the recorded binding reaction, or the time and/or location when the recorded binding interaction occurred.

As used herein, the terms “label” and “detectable label” refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some of the implementations described herein, the terms “linker” and “linker moiety” can refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle, such as a structured nucleic acid particle.

In some of the implementations described herein, the term “measurement outcome” can refer to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome includes an empirical or putative measurement outcome for a candidate analyte (e.g. for a candidate protein) that is known or suspected of being present in a sample or assay. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

In some of the implementations described herein, the term “nucleic acid origami” can refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some of the implementations described herein, the term “post-translational modification” can refer to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

In some of the implementations described herein, the term “promiscuous,” when used in reference to a reagent, can mean that the reagent is known or suspected to react with a variety of different analytes in a given sample. For example, an affinity reagent that is known or suspected to recognize a variety of different analytes (e.g. a variety of proteins having different primary sequences) is promiscuous. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample.

In some of the implementations described herein, the terms “protein” and “polypeptide” can refer synonymously to a molecule comprising two or more amino acids joined by a peptide bond. A protein may also be referred to as a polypeptide, oligopeptide or peptide. A protein can be a naturally occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. A protein may have a known biological activity or function. A protein can refer to a full-length or intact sequence of amino acids, as translated from a gene of an organism, or a spliced variant thereof. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state. In some of the implementations described herein, the term “peptide” may refer to a short polypeptide (e.g., containing no more than about 50, 40, 30, 25, 20, 15, 10, or less than 10 amino acid residues). A peptide may be a fragment of a full-length protein. A peptide may be naturally occurring or synthetic. A peptide may have a biological activity or function.

In some of the implementations described herein, the term “protein substrate” can refer to a molecule or ligand that can be transiently bound to a binding site of a protein molecule. A protein substrate may be altered by the catalytic activity of an enzyme molecule. A protein substrate may be an organic molecule, an inorganic molecule, or a combination thereof. A protein substrate may be a naturally occurring molecule or a synthetic molecule. A protein substrate does not need to be modified by an enzyme molecule to be considered a substrate of the enzyme molecule. Any molecule that binds a binding site of a protein for a long enough time period to be observed as bound to the binding site may be considered a protein substrate.

In some of the implementations described herein, the term “single,” when used in reference to an object such as an analyte, can mean that the object is individually manipulated or distinguished from other objects. A single analyte can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some of the implementations described herein, the term “single-analyte resolution” can refer to the detection of, or ability to detect, an analyte on an individual basis, for example, as distinguished from its nearest neighbor in an array.

In some of the implementations described herein, the term “solid support” can refer to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) are attached.

In some of the implementations described herein, the term “structurally unique” can refer to two or more molecules containing differing compositions or spatial arrangements of atoms and/or bonds joining the atoms. Isomers, such as n-butane and isobutane, can be considered structurally differing molecules with respect to the arrangement of covalent bonds between atoms. Likewise, spatial isomers, such as R- and S-chiral isomers or cis- or trans-isomers, can be considered structurally unique. Butane and pentane can be considered structurally differing molecules with respect to the composition and quantity of atoms contained in each respective molecule. Structurally differing molecules should not be construed as necessarily behaving differently with respect to binding with an enzyme molecule; two or more structurally differing molecules may be individually capable of binding to an active site of an enzyme molecule.

In some of the implementations described herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, an increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some of the implementations described herein, the term “tag” can refer to a polymer sequence, such as a nucleic acid sequence or peptide sequence, that is encoded with information that uniquely identifies an object with which it is associated. A tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The tag can be, for example, DNA, RNA, peptides, polysaccharides, or analogs thereof. The length of the tag sequence can be at least about 5, 8, 10, 15, 20, 25, 30, 40, 50, 75, 100 or more residues. Alternatively or additionally, the length of the tag sequence can be at most about 100, 75, 50, 40, 30, 25, 20, 15, 10, 8, 5 or fewer residues.

In some of the implementations described herein, the term “type,” when used in reference to a subset of analytes, can refer to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

In some of the implementations described herein, the term “unique identifier” can refer to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least one analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces, etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

In some of the implementations described herein, the term “unique identifier label” can refer to a unique identifier that is a particle, molecule or moiety that provides a detectable characteristic. The detectable characteristic can be, for example, an optical signal such as absorbance of radiation, luminescence (e.g. fluorescence) emission, luminescence lifetime, luminescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

In some of the implementations described herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g. in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel. A vessel can be made from multiple materials, for example, including a well in a solid support that is covered by a seal such as a wax or fluid that is immiscible with fluid in the well.

The embodiments set forth below and recited in the claims can be understood in view of the above definitions.

### Detectable Probes for Enzyme Identification and Characterization

In an aspect, provided herein is a method, comprising: (a) providing a plurality of polypeptides, wherein each polypeptide of the plurality of polypeptides is individually co-localized with a first unique identifier, and wherein each polypeptide of the plurality of polypeptides has an unknown identity, (b) contacting to the plurality of polypeptides a plurality of binding reagents, wherein each binding reagent comprises a same protein substrate or enzymatic cofactor, and wherein the protein substrate or enzymatic cofactor is attached to a second unique identifier, (c) detecting for each polypeptide of the plurality of polypeptides presence or absence of a co-localized signal from the second unique identifier with the first unique identifier of the polypeptide, and (d) for each polypeptide with the co-localized signal from the second unique identifier with the first unique identifier, assigning a protein identity to the polypeptide.

In another aspect, provided herein is a method, comprising: (a) contacting a protein molecule to a binding reagent in the presence of a molecular activator, wherein the protein molecule is co-localized with a first unique identifier, wherein the binding reagent comprises a protein substrate, and wherein the protein substrate is attached to a second unique identifier, and (b) detecting a co-localized signal from the second unique identifier of the binding reagent and the first unique identifier of the polypeptide, thereby identifying binding of the protein substrate to the protein molecule.

Systems, compositions, and methods set forth herein may be exemplified with enzymes as analytes. The skilled person will readily recognize that the systems, compositions, and methods may readily be modified to include any protein that is configured to bind a protein substrate or enzymatic cofactor, as set forth herein.

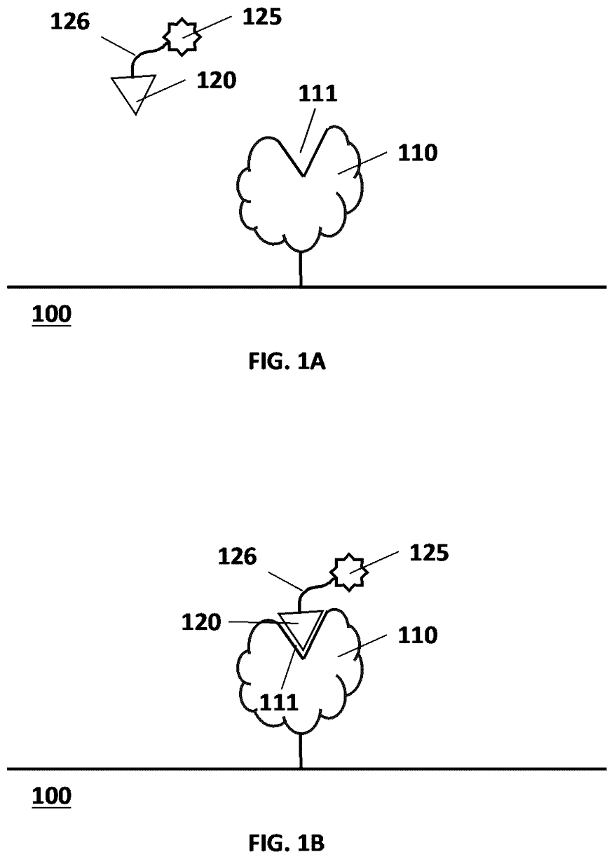

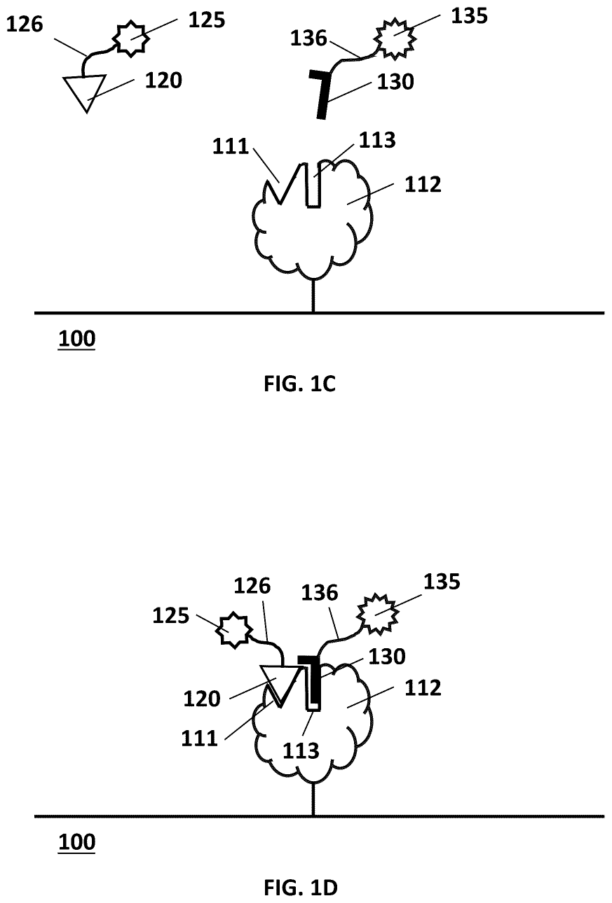

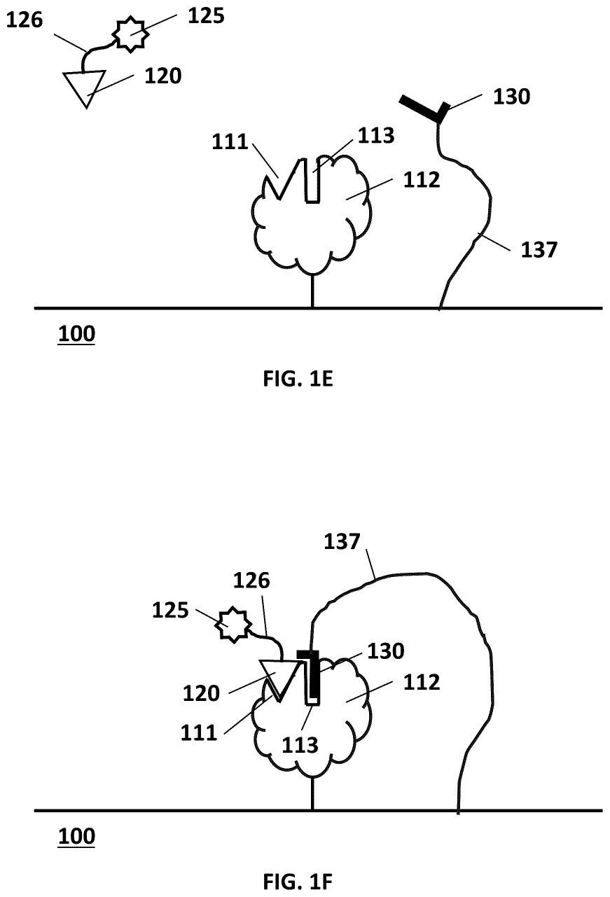

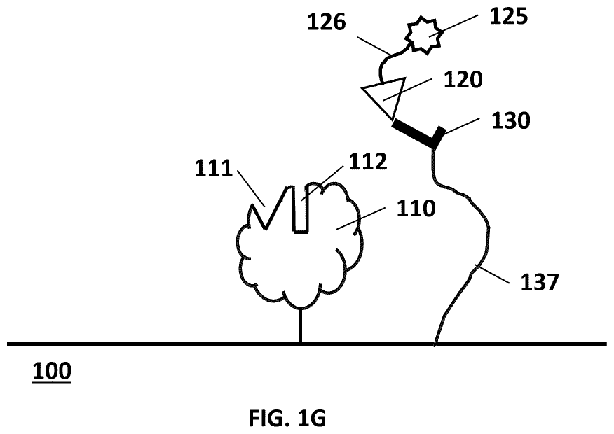

FIGS. 1A-1G depict configurations of systems for identifying enzyme analytes with binding reagents comprising protein substrates or enzymatic cofactors. FIG. 1A depicts an enzyme molecule 110, optionally bound to a solid support 100. The enzyme molecule 110 comprises a substrate or cofactor binding site 111. The enzyme molecule 110 is contacted with a binding reagent comprising a protein substrate or enzymatic cofactor 120 that is attached to a detectable label 125 (e.g., a fluorophore, a luminophore, a radiolabel, a spin label, a barcode, etc.) by a linking moiety 126 (e.g., a particle, a polymer strand, a nucleic acid nanoparticle, etc.). As shown in FIG. 1B, the protein substrate or enzymatic cofactor 120 is bound to the binding site 111 of the enzyme molecule 110, thereby facilitating detection of the binding interaction between the binding reagent and the enzyme molecule 110 (e.g., by optical detection of a signal from the detectable label 125 at the address of the solid support 100 that is attached to the enzyme molecule 110).

FIGS. 1C-1D depicts similar configurations to FIGS. 1A-1B , with an enzyme molecule 112 that contains two protein substrate or enzymatic cofactor binding sites 111 and 113. The enzyme molecule 112 is contacted with the first binding reagent containing the protein substrate or enzymatic cofactor 120, and the second binding reagent containing a protein substrate or enzymatic cofactor 130. The second binding reagent further comprises a second detectable label 135 that is attached to the second protein substrate or enzymatic cofactor 130 by a linking moiety 136. As shown in FIG. 1D, the first binding reagent and second binding reagent are each respectively bound to the enzyme molecule 112 by binding of the first protein substrate or enzymatic cofactor 120 to the first binding site 111 and by binding of the second protein substrate or enzymatic cofactor 130 to the second binding site 113. Binding of both binding reagents to the enzyme molecule 112 may be detected by detecting co-localization of detectable labels 125 and 135, for example at the same address of the solid support 100. Alternatively, co-localization of detectable labels 125 and 135 can be detected by a method such as fluorescence resonance energy transfer (FRET), fluorescence quenching, or reduction of anisotropy of either or both detectable labels 125 and 135.

FIGS. 1E-1G depicts a system comprising a tethered binding reagent. FIG. 1E depicts a system with a similar configuration to that of FIG. 1C, however the second protein substrate or enzymatic cofactor 130 is immobilized to the solid support 100 by a linking moiety 137. FIG. 1F depicts a second configuration in which the first protein substrate or enzymatic cofactor 120 and the second protein substrate or enzymatic cofactor 130 have bound to the enzyme molecule 112, thereby facilitating a reaction between the first protein substrate or enzymatic cofactor 120 and the second protein substrate or enzymatic cofactor 130. FIG. 1G depicts a third configuration of the system, in which the first protein substrate or enzymatic cofactor 120 has been attached to the second protein substrate or enzymatic cofactor 130, thereby co-localizing the detectable label 125 of the first binding reagent at the address of the solid support 100.

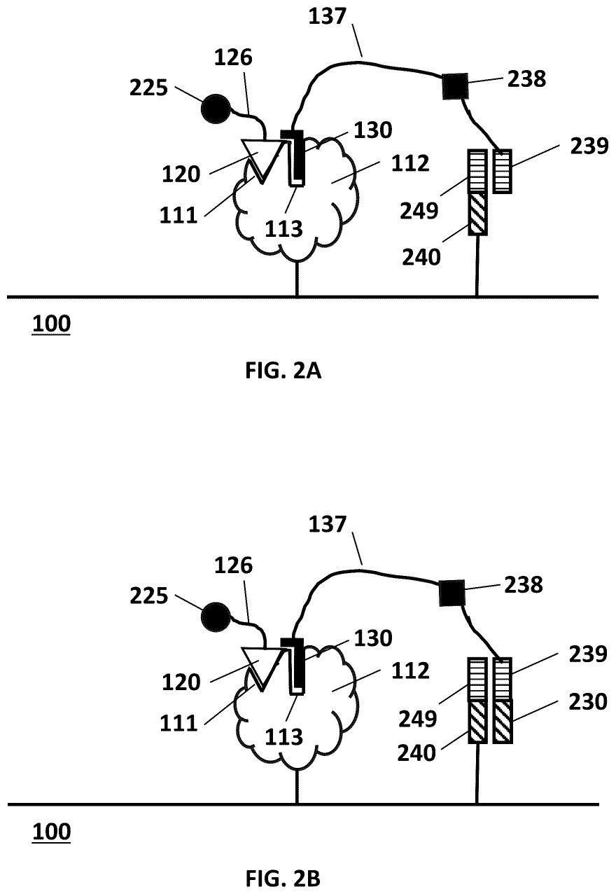

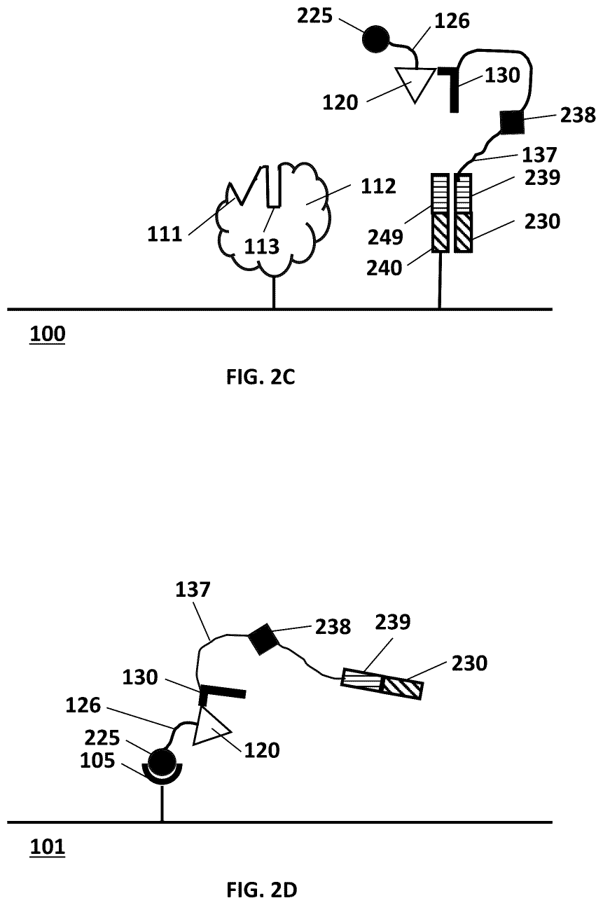

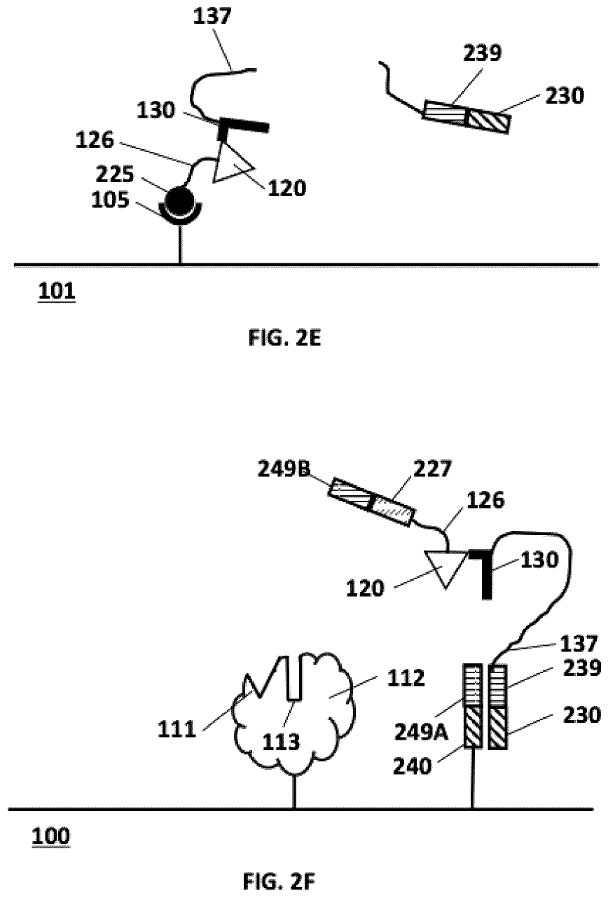

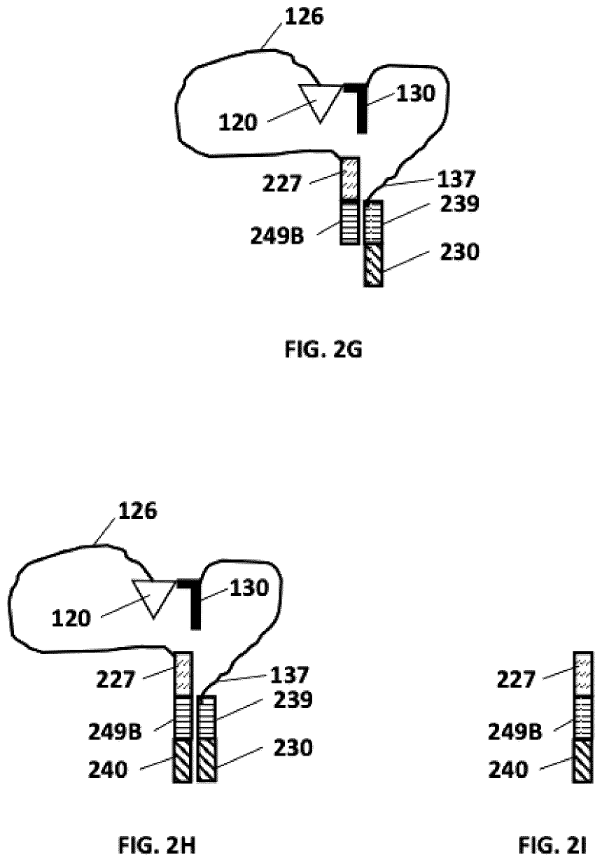

FIGS. 2A-2D depict a system for non-optically detecting a binding interaction between one or more protein substrates or enzymatic cofactors and an enzyme molecule. The system is similar to the system depicted in FIG. 1F with certain modifications. The first binding reagent comprises a detectable label 225 that does not produce a detectable signal (e.g., a barcode moiety, an affinity tag, a purification tag, a binding ligand such as biotin, etc.). Further, the enzyme molecule 112 is co-localized on the substrate 100 with an analyte barcode moiety 240 that contains a sequence that distinguishes the enzyme molecule from any other present analytes. As depicted, the barcode moiety 240 and the enzyme molecule 112 are each immobilized at a same address of a solid support 100. Alternatively, the analyte barcode moiety 240 and the enzyme molecule 112 may be attached to each other or co-localized on a same particle (e.g., a nucleic acid particle, a polymer particle, etc.). The analyte barcode moiety 240 is attached to a capture moiety 249 that couples to a complementary capture moiety 239 of the linking moiety 137 of the second binding reagent. In one embodiment, the capture moiety 249 and the complementary capture moiety 239 comprises oligonucleotides with homologous nucleotide sequences. Optionally, the linking moiety 137 that attaches the second protein substrate or enzymatic cofactor 130 to the complementary capture moiety 239 comprises a cleavable moiety 238 (e.g., a photocleavable functional group, a nucleic acid cleaving site for a restriction enzyme, a peptide cleaving site for a protease, etc.).

FIG. 2B depicts a configuration of the system in which the complementary capture moiety 239 has been extended by a polymerase extension reaction (e.g., by contacting the complex with a polymerase enzyme), thereby attaching a complementary barcode moiety 230 to the complementary capture moiety 239.

FIG. 2C depicts a third configuration in which the first binding reagent and the second binding reagent are attached to each other and have been released from the enzyme molecule 112. The complex of the first binding reagent and the second binding reagent remains co-localized with the enzyme molecule 112 by coupling of the capture moiety 249 to the complementary capture moiety 239.

FIG. 2D depicts a fourth configuration in which the complex comprising the first binding reagent and the second binding reagent has been released from the first solid support 100 and transferred to a second solid support 101 comprising a complementary binding moiety 105. The complementary binding moiety 105 has bound to the detectable label 225 of the first binding reagent, thereby immobilizing the complex containing the first binding reagent and the second binding reagent on the second solid support 101.

FIG. 2E depicts a fifth configuration, in which the cleavable moiety 238 has been severed, thereby detaching the complementary capture moiety 239 and the complementary barcode moiety 230 from the complex comprising the first binding reagent and the second binding reagent. Subsequently, the complementary barcode moiety may be detected by a sequencing device, thereby detecting the presence of the binding of the first binding reagent and the second binding reagent to the enzyme molecule 112. Although depicted with two binding reagents, the system of FIGS. 2A-2E is adaptable to a single binding reagent by binding between a complementary capture moiety 239 of the binding reagent (which acts as a barcode moiety for the binding reagent) with the capture moiety 249 of the analyte barcode moiety 240.

FIGS. 2F-2I depict an alternative configuration for non-optically detecting a binding interaction between one or more protein substrates or enzymatic cofactors and an enzyme molecule. FIG. 2F depicts a similar configuration to FIG. 2C, with the first binding reagent comprising a barcode moiety 227 attached to a second capture moiety 249B having the same sequence as the capture moiety 249A attached to the analyte barcode 240. FIG. 2G depicts a dissociated barcode moiety complex after the complementary barcode moiety 230 has been formed. The second capture moiety 249B can hybridize to the complementary capture moiety 239, thereby forming a partially double-stranded nucleic acid that can be extended by a polymerase extension reaction. FIG. 2H depicts a configuration after a polymerase extension reaction, in which the analyte barcode moiety 240 has been extended onto the second capture moiety 249B. An interaction identification moiety has been formed that contains information from both binding reagent barcodes and the analyte barcode. FIG. 2I depicts a cleaved interaction identification moiety that may be subsequently sequenced to detect the interaction of both binding reagents with the enzyme molecule.

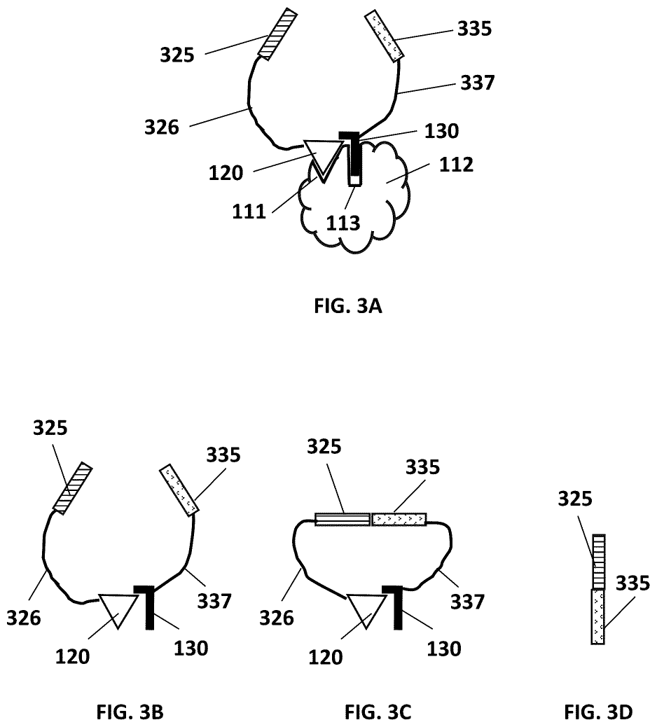

FIGS. 3A-3D illustrate a system that utilizes a ligation-based approach to detecting binding of one or more protein substrates or enzymatic cofactors to an enzyme molecule. The approach is depicted without a solid support, although the system may be readily adapted to include features depicted in FIGS. 1A-1G and/or FIGS. 2A-2E. As shown in FIG. 3A, the enzyme molecule 112 has bound a first binding reagent at its first binding site 111 and a second binding reagent at its second binding site 113. The first binding reagent comprises a first protein substrate or enzymatic cofactor 120 attached to a first binding reagent barcode moiety 325 by a first linking moiety 326. The second binding reagent comprises a second protein substrate or enzymatic cofactor 130 attached to a second binding reagent barcode moiety 335 by a second linking moiety 337. Optionally, the first linking moiety 326 and the second linking moiety 337 may comprise a cleavable moiety (not shown).

FIG. 3B depicts a second configuration comprising a complex formed by the attachment of the first binding reagent to the second binding reagent by the enzyme molecule 112 after the complex has been released from the enzyme molecule 112. FIG. 3C depicts a third configuration formed by ligation of the first binding reagent barcode moiety 325 to the second binding reagent barcode moiety 335 (e.g., nucleic acid ligation, peptide ligation, etc.). FIG. 3D depicts an optional fourth configuration in which the ligated product of the first binding reagent barcode moiety 325 and the second binding reagent barcode moiety 335 are detached from the complex containing the first binding reagent and the second binding reagent (e.g., by cleavage of cleavable moieties of the respective linking moieties 326 and 337). The ligated product may be detected by a sequencing device, thereby detecting the interaction of the first binding reagent and the second binding reagent with the enzyme molecule 112.

The system depicted in FIGS. 3A-3D facilitates the detection of binding interactions of the enzyme molecule, but does not provide information on which enzyme molecule mediated the attachment of the binding reagents. Modification of the ligation-based system to include a barcode moiety associated with the enzyme molecule can facilitate the detection of which enzyme molecule mediated the attachment of the binding reagents. If an enzyme molecule only interacts with one protein substrate or enzymatic cofactor, a barcode moiety for a binding reagent containing the one protein substrate or enzymatic cofactor may be directly attached to a barcode moiety for the enzyme molecule to facilitate detection of the binding interaction of the enzyme molecule with the one protein substrate or enzymatic cofactor. Additional aspects of non-optical protein detection and characterization are provided in U.S. patent application Ser. No. 19/338,819, which is herein incorporated by reference in its entirety.

Systems and methods disclosed herein may utilize binding reagents comprising at least one protein substrate or enzymatic cofactor. A binding reagent can comprise two or more protein substrates or enzymatic cofactors. A plurality of protein substrates or enzymatic cofactors can include two or more copies of a structurally identical protein substrate or enzymatic cofactor. For example, a binding reagent may comprise two or more copies of a protein substrate, with each copy of the protein substrate attached to the binding reagent by the same functional group of the protein substrate. In another example, a binding reagent may comprise two or more copies of a protein substrate, with each copy of the protein substrate attached to the binding reagent by a different functional group of the protein substrate. A plurality of protein substrates or enzymatic cofactors can include two or more structurally unique protein substrates or enzymatic cofactors. For example, a binding reagent for a tRNA synthetase may comprise an attached amino acid and an attached transfer RNA comprising a codon that corresponds to the attached amino acid. Binding reagents comprising two or more binding moieties are described in U.S. Pat. No. 11,692,217 and U.S. Patent Publication US20230090454A1, each of which is herein incorporated by reference in its entirety.

A protein substrate or enzymatic cofactor may be incorporated into a binding reagent by attachment to a linking moiety. A linking moiety may facilitate attachment between the protein substrate or enzymatic cofactor and another binding reagent component (e.g., detectable label, tether strand, etc.). A linking moiety may provide spatial separation between a protein substrate or enzymatic cofactor and another binding reagent component.

A linking moiety may comprise a polymer strand. In some cases, a linking moiety can comprise a linear polymer strand. Alternatively, a linking moiety can comprise a branched polymer strand. A polymer strand can include a biopolymer strand (e.g., an oligonucleotide, a peptide strand, a polysaccharide, etc.), a synthetic polymer (e.g., polyethylene glycol, polyethylene, polypropylene, polymethyl methacrylate, etc.), or a combination thereof.

A polymer strand of a linking moiety may comprise a sequence of bonded residues or monomers. Linking moieties comprising biopolymers can include naturally-occurring monomers or residues (e.g., nucleotides, amino acids, saccharides, etc.) as well as modified versions thereof (e.g., LNAs, PNAs, non-natural amino acids, etc.). A polymer strand of a linking moiety may comprise a sequence of at least about 2, 3, 4, 5, 10, 15, 20, 30, 40, 50, 75, 100, 150, 200, 250, 300, 400, 500, or more than 500 bonded monomers or residues. Alternatively or additionally, a polymer strand of a linking moiety may comprise a sequence of no more than about 500, 400, 300, 250, 200, 150, 100, 75, 50, 40, 30, 20, 15, 10, 5, 4, 3, or less than 3 bonded monomers or residues.

A polymer strand of a linking moiety may be configured to facilitate binding of an attached protein substrate or enzymatic cofactor to a protein molecule. In particular, the portion of a polymer strand adjacent to an attached protein substrate or enzymatic cofactor may be engineered to facilitate favorable interactions (e.g., electrostatic attraction, ion-mediated charge bridging, etc.) with a protein molecule and/or inhibit unfavorable interactions with the protein molecule (e.g., electrostatic repulsion, steric repulsion, etc.). A portion of a polymer strand that is attached to a protein substrate or enzymatic cofactor may comprise one or more structural features that affect the conformation, electrostatic interactions, or steric interactions of the polymer strand with a protein molecule to which the protein substrate or enzymatic cofactor may bind.

A polymer strand of a linking moiety may be configured to inhibit formation of ordered or non-random structures. For example, a nucleic acid strand may comprise an oligonucleotide sequence with no internal complementarity, thereby inhibiting self-annealing of the nucleic acid strand to itself. In another example, a peptide strand may be designed to have minimal secondary structures, such as alpha helices or beta sheets. Examples of peptide design with minimal ordered structure are set forth in U.S. Pat. No. 8,673,860, which is herein incorporated by reference in its entirety. A polymer strand provided to a linking moiety, such as a biopolymer or a synthetic polymer, may be configured to have a globular or random spatial configuration in a fluidic medium. Preferably, a polymer strand provided to a linking moiety can be labile (e.g., a first moiety of the polymer strand can diffuse independently of a second non-contiguous moiety of the polymer strand). Methods of designing polymer strands with minimal ordered or non-random structure are known in the art. Computational tools (e.g., oligonucleotide designers, protein folding tools, helical peptide analysis, etc.) may be utilized to design sequenced polymers.

Alternatively, a polymer strand of a linking moiety may be configured to facilitate formation of ordered or non-random structures. A polymer strand provided to a linking moiety may be configured to form a stable secondary or tertiary structure (e.g., a helix, a beta sheet, a stem-loop structure, etc.). A polymer strand provided to a linking moiety may comprise a first moiety of the polymer strand coupled to a second moiety of the polymer strand, in which the first moiety and the second moiety are non-contiguous in the residue sequence of the polymer strand. In some configurations, a polymer strand provided to a linking moiety may form a binding interaction with a protein substrate or enzymatic cofactor attached to the linking moiety. The binding specificity of a binding reagent may be increased by such a configuration. Preferably, the binding interaction of the protein substrate or enzymatic cofactor for the linking moiety is weaker than the binding interaction of the protein substrate or enzymatic cofactor for an active site of a protein molecule. In such cases, the protein substrate or enzymatic cofactor of the binding reagent is most likely to decouple from the linking moiety of the binding reagent if interacting with a protein molecule having a binding specificity for the protein substrate or enzymatic cofactor.

Many protein substrates and/or enzymatic cofactors are small molecules with a reduced surface area to support both enzyme binding and label attachment. Accordingly, prior information and molecular details of the binding interaction between a protein binding site and a substrate or cofactor can be useful in guiding the modification of the substrate or cofactor or design of a linking moiety so that a detectable label can be added without interfering with the binding to the protein binding site. Such prior information about the molecular details of the binding site, substrate or cofactor structure, or bound conformations between the two entities can come from experimental data (e.g., x-ray crystallography, nuclear magnetic resonance, CryoEM, structure-activity relationship analysis, etc.), from predicted modeling data (e.g., computational tools for docking or defining protein-protein interactions), and/or from an understanding of the biology and biological mechanism of the cofactor (i.e., which portions of the cofactor are involved in enzymatic interactions and which portions are not involved in enzymatic interactions and where the addition of the detectable label is likely to be compatible with substrate or cofactor binding to the binding site).

A portion of a polymer strand adjacent to an attached protein substrate or enzymatic cofactor may comprise a peptide. The peptide portion of a linking moiety may be substantially devoid of any secondary or tertiary structure. Alternatively, a peptide portion of a linking moiety may form a secondary or tertiary structure that is known to interact with another protein. A portion of a polymer strand adjacent to an attached protein substrate or enzymatic cofactor may comprise one or more hydrophobic residues or monomers. A portion of a polymer strand adjacent to an attached protein substrate or enzymatic cofactor may comprise one or more hydrophilic residues or monomers.

A portion of a polymer strand adjacent to an attached protein substrate or enzymatic cofactor may comprise a moiety that is configured to spatially orient the attached protein substrate or enzymatic cofactor relative to the protein molecule or another portion of a binding reagent. A portion of a polymer strand adjacent to an attached protein substrate or enzymatic cofactor may comprise a rigid moiety, such as double-stranded DNA or an unsaturated hydrocarbon moiety (e.g., an alkenyl moiety, an alkynyl moiety, etc.). An unsaturated hydrocarbon moiety may be configured in a cis- or trans-conformation depending upon a desired orientation of an attached protein substrate or enzymatic cofactor. A portion of a polymer strand adjacent to an attached protein substrate or enzymatic cofactor may comprise a flexible moiety, such as a single-stranded nucleic acid, a peptide, or a polyethylene glycol (PEG) moiety. In some cases, a polymer chain may comprise a chiral center. In particular cases, an attached protein substrate or enzymatic cofactor may comprise a first chiral center and a polymer strand attached to the protein substrate or enzymatic cofactor may comprise a second chiral center. The relative configurations of the first chiral center and the second chiral center may be chosen to provide a particular configuration of the attached protein substrate or enzymatic cofactor relative to a protein molecule or another component of a binding reagent.

The conformation of a polymer strand may depend upon the physical environment contacting the polymer strand. A polymer strand may have a coiled or condensed configuration, a linear configuration, or a combination thereof, depending upon the surrounding physical or chemical environment. The configuration of a polymer strand may depend upon the chemical composition of the polymer strand as well as the chemical composition (e.g., buffer composition, ion composition, ionic strength, pH, etc.) of a fluid containing the polymer strand. A binding reagent may be provided in a fluidic medium that is configured to facilitate the activity of a protein molecule. Accordingly, a polymer strand may be configured to contain a region of coiled or condensed structure or a region of linear or extended structure in the fluidic medium that is configured to facilitate the activity of a protein molecule. Methods for formulating fluidic media to facilitate enzymatic activity are known in the art. The skilled person will recognize polymer strand compositions that may provide a desired conformation depending upon the chosen fluid composition.

In some cases, a binding reagent may comprise a linking moiety comprising a particle, such as a nanoparticle or microparticle. A binding reagent component (e.g., a protein substrate, an enzymatic cofactor, a detectable label, a tether strand, etc.) may be attached to a particle by a polymer strand. A linking moiety may comprise a particle comprising a polymer nanoparticle, a dendrimer, a nucleic acid nanoparticle, an organic nanoparticle, an inorganic nanoparticle, or a combination thereof. Useful particle compositions and systems for forming binding reagents are provided in U.S. Pat. No. 11,692,217, and U.S. Patent Publication No. 20240280568A1, each of which is herein incorporated by reference in its entirety.

A protein substrate or enzymatic cofactor may be attached to a binding reagent or a linking moiety thereof by a covalent bond between a first functional group of the protein substrate or enzymatic cofactor and a second functional group of the binding reagent or the linking moiety. A protein substrate or enzymatic cofactor may comprise n unique functional groups that can facilitate attachment by covalent bonding, hereinafter denoted as Rₙ (i.e., R₁, R₂, R₃, etc.). For example, the amino acid tyrosine contains an amine functional group and a carboxyl functional group, as well as multiple potentially reactive sites on its phenolic sidechain. In some cases, only a portion of a protein substrate or enzymatic cofactor may interact with an enzymatic binding site, providing a subset of the Rₙ functional groups external to the enzymatic binding site when the protein substrate or enzymatic cofactor is bound. A functional group R of the subset of the Rₙ functional groups may be chosen for attachment to a binding reagent or a linking moiety thereof. In some cases, covalent binding of a protein substrate or enzymatic cofactor to a functional group of a binding reagent or a linking moiety thereof may form a chiral center at the first bonded atom of the binding reagent or linking moiety.

Methods and systems set forth herein may be configured to assay populations of unknown analytes to identify and/or characterize individual enzyme molecules that may be present in the sample. Single-molecule methods may be particularly advantageous. A single-molecule method may comprise a method that provides each individual analyte of a population of analytes with a single unique identifier that facilitates distinguishing the individual analyte from any other analyte of the population of analytes. A unique identifier for an analyte may be a spatially-unique identifier. In some configurations, a population of analytes may be immobilized on a solid support, in which the solid support contains a plurality of sites, in which each site of the plurality of sites contains no more than one analyte of the population of analytes, and in which each site is optically resolvable (e.g., separated by an optically resolvable distance) from any other site of the plurality of sites. Accordingly, observation of a binding interaction (e.g., via detection of optical signals) at an individual address of the solid support can be utilized to identify and/or characterize the single analyte present at the individual address.

Alternatively, a unique identifier for an individual analyte may be a coded moiety such as a nucleic acid barcode or a peptide barcode. Each individual analyte of a population of analytes may be attached to or otherwise associated with a barcode moiety that facilitates the distinguishing of the individual analyte from any other analyte of the population of analytes. Use of barcode moieties to distinguish individual analytes may be amenable to both array-based methods and fluid-phase methods. If a population of analytes is immobilized on a solid support, in which each individual analyte is co-localized at an address of the solid support with a unique barcode moiety, the individual analytes may or may not be separated from any other analyte by a spatially-resolvable distance.

In some cases, a protein substrate or enzymatic cofactor may be specific to a particular enzyme or other ligand-binding protein. For example, a metabolic intermediate may be a substrate for a single enzyme that transforms the metabolic intermediate into a second metabolic intermediate or a product. Accordingly, a binding reagent comprising the specific protein substrate or enzymatic cofactor may bind specifically to only the protein of which it is a substrate or cofactor. In other cases, a protein substrate or enzymatic cofactor may be promiscuous (i.e., binding to two or more differing enzymes). For example, certain substrates or cofactors, such as ATP, NADH, NADPH, etc. are utilized in numerous enzymes within a proteome of a given organism. Likewise, certain protein substrates or enzymatic cofactors may be utilized in secondary or currently unknown interactions aside from their known function. For example, transfer RNAs may be utilized as molecular regulators in certain functions, including peptidoglycan synthesis and cellular signaling. Accordingly, a binding reagent comprising a protein substrate or enzymatic cofactor may bind to two or more structurally differing enzymes.

A protein molecule may bind to two or more protein substrates and/or enzymatic cofactors. In some cases, a protein molecule may bind to two or more differing protein substrates and/or enzymatic cofactors. In some cases, a protein molecule may bind to two or more of the same protein substrates and/or enzymatic cofactors. Accordingly, two or more binding reagents, as set forth herein, may be bound to a protein molecule. A method may comprise a step of detecting two or more binding reagents co-localized with a protein molecule. Detecting two or more binding reagents co-localized with a protein molecule may comprise detecting co-localization of a first unique identifier associated with a protein molecule with a second unique identifier associated with a first binding reagent and a third unique identifier associated with a second binding reagent.

Binding of two or more protein substrates or enzymatic cofactors to a protein molecule may occur in a sequential manner. For example, binding of a second protein substrate or enzymatic cofactor may be dependent upon an allosteric conformation change of a protein molecule caused by binding of a first protein substrate or enzymatic cofactor. Detection of two or more binding reagents, as set forth herein, bound to a single enzyme molecule may be dependent upon providing the two or more binding reagents in a sequential manner. Accordingly, a method of identifying a protein molecule may comprise the steps of: i) contacting a fluidic medium comprising a first binding reagent and a second binding reagent to a protein molecule, and ii) detecting co-localization of a first unique identifier associated with the protein molecule with a second unique identifier associated with the first binding reagent and a third unique identifier associated with the second binding reagent. Alternatively, a method of characterizing a protein molecule may comprise the steps of: i) contacting a first fluidic medium comprising a first binding reagent to a protein molecule, ii) contacting a second fluidic medium comprising a second binding reagent to the protein molecule, and iii) detecting presence or absence of co-localization of a first unique identifier associated with the protein molecule with a second unique identifier associated with the first binding reagent and/or a third unique identifier associated with the second binding reagent. If co-localization of only the second introduced binding reagent is observed, a method may further comprise the steps of: iv) providing a third fluidic medium comprising the first binding reagent, and v) detecting presence or absence of co-localization of a first unique identifier associated with the protein molecule with a second unique identifier associated with the first binding reagent. Variations in sequencing and detection of unique identifiers may facilitate characterization of the binding sequence of a protein molecule.

If a system is configured to detect binding interactions by an optical or other spatial detection method (e.g., detecting at single-molecule resolution on a solid support), detecting co-localization of a first unique identifier associated with a protein molecule with a second unique identifier associated with a first binding reagent and a third unique identifier associated with a second binding reagent may comprise detecting a first signal from the second unique identifier (e.g., a fluorophore, a luminophore, a radiolabel, a spin label) and a second signal from the third unique identifier (e.g., a fluorophore, a luminophore, a radiolabel, a spin label) at a same address of a solid support. If a protein molecule binds two or more of the same protein substrate or enzymatic cofactor, the first signal and the second signal may be additive. Accordingly, a quantity of binding reagents having the same protein substrate or enzymatic cofactor may be determined by step changes in signal magnitude at an address of a solid support if each binding reagent contains the same detectable label. If a protein molecule binds two or more differing protein substrates or enzymatic cofactors, the first signal and the second signal may be distinguishably detected co-localized (e.g., different emission wavelengths detected at a same address of a solid support; different barcode sequences in a same interaction identification moiety).

Binding patterns of two or more binding reagents, as set forth herein, may also be useful for identifying two or more differing proteins. Promiscuous binding reagents (e.g., binding reagents comprising protein substrates or enzymatic cofactors that bind to two or more differing enzyme molecules) may be useful for identifying protein molecules with a common binding specificity for a protein substrate or enzymatic cofactor. Subsequent binding by higher specificity binding reagents (e.g., binding reagents containing a protein substrate or enzymatic cofactor for a single enzyme) may facilitate differentiation of differing protein molecules. For example, a first protein molecule may be provided a first identity if it binds a first binding reagent and a second binding reagent, and a first protein molecule may be provided a second identity if it binds the first binding reagent and does not bind the second binding reagent.

An active enzyme molecule may be identified and/or characterized by observing a reaction catalyzed by the enzyme molecule at single-molecule resolution. In some cases, the catalytic activity of a protein may result in the dissociation of a product from the enzyme molecule. If a binding reagent is modified by the activity of a protein molecule, the binding reagent may be dissociated from the enzyme molecule before the binding of the binding reagent to the enzyme molecule can be detected. Accordingly, it may be preferable to provide one or more moieties that facilitate co-localization of a binding reagent with a protein molecule even if dissociation of the binding reagent from the enzyme molecule occurs. Systems containing tethers and dockers, as set forth herein, may be particularly useful for facilitating retention of a binding reagent in proximity to an analyte. In some configurations (e.g., FIGS. 2A-2I), a barcode moiety, such as an analyte-associated barcode moiety, may be configured to facilitate a docker-tether interaction. Systems of tethers and dockers are discussed in more detail in U.S. patent application Ser. No. 18/748,558, which is herein incorporated by reference in its entirety.

In some cases, a protein substrate may need to be activated to facilitate binding of the protein substrate to a protein molecule. Activation of a protein substrate can include the addition of a moiety to the protein substrate or an alteration of the protein substrate into a more reactive form. Activation of a protein substrate may facilitate binding of the protein substrate to a binding site of a protein molecule. A method may further comprise a step of attaching a molecular activator to a protein substrate. In some cases, attaching a molecular activator to a protein substrate may occur in a fluidic medium. In such cases, a method may further comprise, after attaching the molecular activator to the protein substrate, binding the binding reagent to the protein molecule. Alternatively, activation of a protein substrate may be facilitated by a protein molecule. In such a case, attaching a molecular activator to the protein substrate can comprise: i) binding the molecular activator to a protein molecule, ii) binding the protein substrate to the protein molecule, and iii) attaching the molecular activator to the protein substrate by the activity of the protein molecule.

In another aspect, provided herein is a method, comprising: (a) providing a library of binding reagents, wherein each binding reagent of the library of binding reagents comprises a protein substrate or enzymatic cofactor attached to a first unique identifier, wherein the library of binding reagents comprises a plurality of structurally-unique binding reagents, each structurally unique binding reagent comprising a differing attachment conformation of the protein substrate or enzymatic cofactor to the first unique identifier, and each structurally unique binding reagent comprising a first unique identifier that differs from the first unique identifier of any other structurally-unique binding reagent, (b) contacting a protein molecule with the library of binding reagents; wherein the protein is co-localized with a second unique identifier, and (c) detecting co-localization of a first unique identifier of a structurally unique binding reagent with the second unique identifier, thereby identifying a portion of the protein substrate or enzymatic cofactor that is bound by the protein molecule.

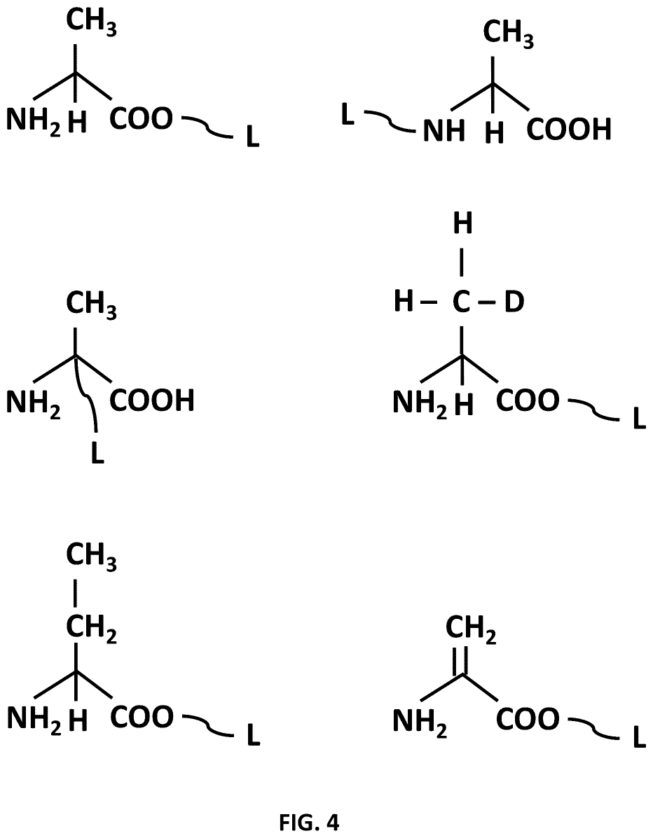

FIG. 4 depicts an exemplary library of binding reagents, with each binding reagent comprising a structurally unique attachment of the protein substrate alanine to a linking moiety L. The upper binding reagents contain alanine attached to the linking moiety L by the carboxyl and amine functional groups, respectively. The center left binding reagent contains alanine attached to the linking moiety L by the center carbon of the amino acid. The remaining library members comprise derivatives of alanine, including a deuterium-substituted derivative (center right), a methylated derivative (lower left), and a dehydrogenated derivative (lower right). Numerous other possible derivatives are not shown but may be included within the scope of the present application.

Members of a library of binding reagents may be contacted to a plurality of copies of a protein molecule to characterize the binding conformation of the protein molecule with a protein substrate and/or enzymatic cofactor. Each member of a library of binding reagents may be contacted to a protein molecule sequentially (i.e., contact a first binding reagent, bind, detect, remove, then repeat with a next binding reagent, etc.). Alternatively, two or more structurally unique binding reagents can be simultaneously contacted to one or more protein molecules if each structurally unique binding reagent is associated with a distinguishable unique identifier.

In some configurations, a library of binding reagents may comprise a plurality of structurally unique binding reagents. Each binding reagent of a plurality of structurally unique binding reagents can contain a same protein substrate or enzymatic cofactor, in each binding reagent is distinguished by the attachment point of the protein substrate or enzymatic cofactor (see, for example, the upper configurations and center left configuration of FIG. 4). In some configurations, a plurality of structurally unique binding reagents can further comprise a derivative of a protein substrate or enzymatic cofactor. A derivative of a protein substrate or enzymatic cofactor may comprise an addition of a functional group, a subtraction of a functional group, a substitution of a functional group, a rearrangement of a chiral center, a change in cis-trans isomerism, or a combination thereof.

For a protein molecule with a known identity, it may be useful to characterize binding interactions of other molecules with the protein at single-molecule resolution. In another aspect, provided herein is a method, comprising: (a) providing a library of binding reagents, wherein each binding reagent of the library of binding reagents comprises a protein binding candidate attached to a first unique identifier, wherein the library of binding reagents comprises a plurality of structurally-unique binding reagents, each structurally unique binding reagent comprising a differing enzyme binding candidate, and each structurally unique binding reagent comprising a first unique identifier that differs from the first unique identifier of any other structurally-unique binding reagent, (b) contacting a protein molecule with the library of binding reagents; wherein the protein is co-localized with a second unique identifier, and (c) detecting co-localization of a first unique identifier of a structurally unique binding reagent with the second unique identifier, thereby identifying a molecule that binds to the protein molecule.

A protein binding candidate may be any conceivable molecule. In some cases, a protein binding candidate can comprise a protein substrate. In some cases, a protein binding candidate can comprise an enzymatic cofactor. In some cases, a protein binding candidate can comprise a derivative of a protein substrate or enzymatic cofactor. In some cases, a protein binding candidate can comprise a pharmaceutical compound. In some cases, a protein binding candidate can comprise a toxin. In some cases, a protein binding candidate can comprise an enzymatic inhibitor or a derivative thereof.

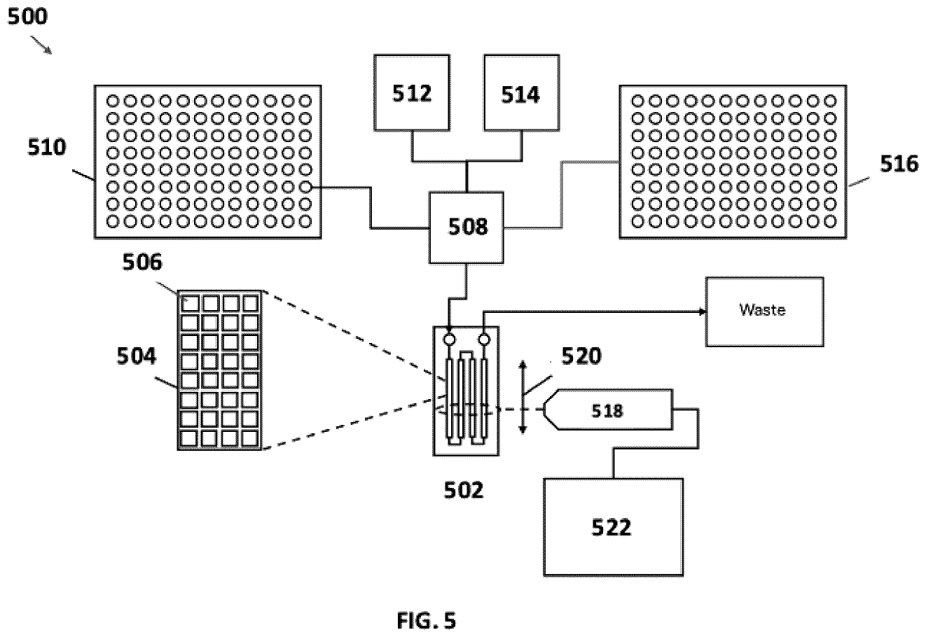

Additionally, provided herein are systems for performing the techniques, reagents, systems, and methods described herein. An example of a system is illustrated in FIG. 5. As shown, the system 500 includes a flowcell 502 that includes an array surface (shown as 504) within the channels of the flow cell upon which individual analyte molecules from a sample may be deposited and immobilized in locations 506 that are individually addressable, and in particular cases are individually optically resolvable from each other using, e.g., fluorescence microscopy or scanning techniques.

The system will also typically include a fluidic delivery system 508 that is configured to deliver different fluids to the flow cell 502 through a series of fluidic lines and utilizing appropriate pumps, valves and other conventional fluid controls. The fluidics delivery system 508 may be fluidically coupled to various sources of fluids and reagents needed to carry out the analysis on the flow cell. For example, as shown, fluidic delivery system 508 is fluidly coupled to a source of a plurality of reagents 510 (shown as a 96 well plate, although any number of different reagent storage systems of varying capacity may be employed) that includes a library of multiple affinity reagents that each have affinity for different characteristics of one or more proteins of interest. Additionally, fluidic delivery system 508 may also be coupled to sources of washing fluids or buffers 512, and removal reagents 514 (for removing bound affinity reagents following detection), as well as any other ancillary fluids and reagents needed for the analysis. Similarly, where flow cells are prepared on the system, the fluidic system may be coupled to sources of different sample materials that are to be analyzed 516 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically include a detection system, such as optical detection system 518, for detecting and recording fluorescent signals arising from different positions on the array surface. Such detection systems may generally include line scanning confocal fluorescent microscope systems, which are capable of scanning across large array surfaces (as shown by arrow 520) to detect and record fluorescence across such surfaces at reasonably high scan rates.

The overall systems also typically include one or more computers or processors 522 for controlling the operation of the instrument system including the fluidic delivery system 508 (e.g., to sample different sample sources 516, reagent sources 510 and delivery timing and volume of each), and detection system 518, among other functions, and for recording the detected signals received from the detection system 518, e.g. fluorescent signals, and analyzing such signals to identify potential binding by each of the different affinity reagents. The computers or processors 522 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, bioRxiv, https://doi.org/10.1101/2021.10.11.463967, U.S. Patent Application No. 2022/0236282, International Patent Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computers or processors 522 can be an electronic device of a detection system, the electronic device being integral to the detection system or remotely located with respect to the detection system. The computers or processors 522 include at least one computer processing unit (CPU, also “processor” and “computer processor” herein), which can be a single core or multi core processor, or a plurality of processors for parallel processing. The computers or processors 522 also include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computers or processors 522 can be operatively coupled to a computer network (“network”) with the aid of the communication interface. The network can be the Internet, an internet and/or extranet, or an intranet and/or extranet that is in communication with the Internet. The network, in some cases, is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 522, can implement a peer-to-peer network, which may enable devices coupled to the computer system 522 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the computers or processors 522 can be included in the circuit. In some cases, the circuit is an application specific integrated circuit (ASIC).

The storage unit can store files, such as drivers, libraries and saved programs. The storage unit can store user data, e.g., user preferences and user programs. The computers or processors 522, in some cases, can include one or more additional data storage units that are external to the computers or processors 522, such as located on a remote server that is in communication with the computers or processors 522 through an intranet or the Internet.

The computers or processors 522 can communicate with one or more remote computer systems through the network. For instance, the computers or processors 522 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computers or processors 522 via the network.

Methods as described herein can be implemented by way of machine (e.g., computer processor) executable code stored on an electronic storage location of the computers or processors 522, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored in memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 522, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computers or processors 522 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination, the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths, such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents' binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produce inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. Nos. 10,473,654 or 11,282,585; US Pat. App. Pub. Nos. 2020/008294A1 or 2023/0114905A1; or Egertson et al., BioRxiv (2021), DOI: 10.1101/2021.10.11.463967, each of which is incorporated herein by reference. It will be recognized that the methods set forth herein that are utilized to decode extant proteins may be useful for other analyte identification assays, provided said analyte identification assays provide a binding profile that can be decoded.

A protein can optionally be detected based on its enzymatic or biological activity. For example, a protein can be contacted with a reactant that is converted to a detectable product by an enzymatic activity of the protein. In other assay formats, a first protein having a known enzymatic function can be contacted with a second protein to determine if the second protein changes the enzymatic function of the first protein. As such, the first protein serves as a reporter system for the detection of the second protein. Exemplary changes that can be observed include, but are not limited to, activation of the enzymatic function, inhibition of the enzymatic function, attenuation of the enzymatic function, degradation of the first protein or competition for a reactant or cofactor used by the first protein. Proteins can also be detected based on their binding interactions with other molecules such as other proteins, nucleic acids, nucleotides, metabolites, hormones, vitamins, small molecules that participate in biological signal transduction pathways, biological receptors or the like. For example, a protein that participates in a signal transduction pathway can be identified as a particular candidate protein by detecting binding to a second protein that is known to be a binding partner for the candidate protein in the pathway.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine the presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g. (strept)avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array is set forth in U.S. Pat. Nos. 11,203,612 and 11,505,796, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

Alternatively to single-analyte resolution, a method can be carried out at ensemble-resolution or bulk-resolution. Bulk-resolution configurations acquire a composite signal from a plurality of different analytes or affinity agents in a vessel or on a surface. For example, a composite signal can be acquired from a population of different protein-affinity agent complexes in a well or cuvette, or on a solid support surface, such that individual complexes are not resolved from each other. Ensemble-resolution configurations acquire a composite signal from a first collection of proteins or affinity agents in a sample, such that the composite signal is distinguishable from signals generated by a second collection of proteins or affinity agents in the sample. For example, the ensembles can be located at different addresses in an array. Accordingly, the composite signal obtained from each address will be an average of signals from the ensemble, yet signals from different addresses can be distinguished from each other.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different analytes can be attached to different unique identifiers (e.g. proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes such as an analytical process used for detecting, identifying, characterizing or quantifying an analyte. Analytes can be attached to unique identifiers via covalent or non-covalent (e.g. ionic bond, hydrogen bond, van der Waals forces etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

A protein or other analyte can be attached to a unique identifier (e.g. an address in an array) using any of a variety of means. The attachment can be covalent or non-covalent. Exemplary covalent attachments include chemical linkers such as those achieved using click chemistry or other linkages known in the art or described in U.S. Pat. Nos. 11,203,612 or 11,505,796 or US Pat. App. Pub. No 2023/0167488 A1, each of which is incorporated herein by reference. Non-covalent attachment can be mediated by receptor-ligand interactions (e.g. (strept)avidin-biotin, antibody-antigen, or complementary nucleic acid strands), for example, in which the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful SNAPs. The use of SNAPs and other moieties to attach proteins to unique identifiers such as tags or addresses in an array are set forth in U.S. Pat. Nos. 11,203,612 or 11,505,796 or US Pat. App. Pub. No 2023/0167488 A1, each of which is incorporated herein by reference.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g. a laser or lamp) then through a window of the vessel. The detection apparatus can further include a camera or other detector that acquires signals transmitted through the window of the vessel and through an optical train. Optionally, excitation and emission can be transmitted through the same optical train; however, separate optical trains can also be useful.

A detection apparatus can include a light-sensing device that is appropriate for detecting a characteristic set forth herein or known in the art. Particularly useful components of a light-sensing device can include, but are not limited to, optical sub-systems or components used in nucleic acid sequencing systems. Examples of useful subsystems and components thereof are set forth in US Pat. App. Pub. No. 2010/0111768 A1 or U.S. Pat. Nos. 7,329,860; 8,951,781 or 9,193,996, each of which is incorporated herein by reference. Other useful light-sensing devices and components thereof are described in U.S. Pat. Nos. 5,888,737; 6,175,002; 5,695,934; 6,140,489; or 5,863,722; or US Pat. Pub. Nos. 2007/007991 A1, 2009/0247414 A1, or 2010/0111768; or WO2007/123744, each of which is incorporated herein by reference. Light-sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g. luminescent detection), one or more analytes (e.g. proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide-assisted excitation, or the like. Optical filters or other optical components can be present to tune the wavelength, polarization or other optical properties of excitation and/or emission radiation used by a luminescence detector.

A light-sensing device may be based upon any suitable technology, and may be, for example, a charge-coupled device (CCD) sensor that generates pixelated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light-sensing devices can optionally be coupled with one or more excitation sources, for example, lasers, light emitting diodes (LEDs), arc lamps or other energy sources known in the art.

A light-sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. Nos. 7,122,482 or 8,765,359, or US Pat. App. Pub. No 2013/0116153 A1, each of which is incorporated herein by reference. The wells can be configured for selective excitation, for example, as set forth in U.S. Pat. Nos. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes, such that the analytes can be quantified as set forth in WO 2020/176793 A1, which is incorporated herein by reference.

A detection apparatus need not be configured for optical detection. For example, an electronic detector can be used for the detection of protons or charged labels (see, for example, US Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in US Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

Accordingly, a coupling chemistry may be selected based upon the criterion that it provides a sufficiently stable coupling of an analyte to a solid support for a time scale that meets or exceeds the time scale of a method as set forth herein. For example, a polypeptide identification method can require a coupling of the analyte to the solid support for a sufficient amount of time to permit a series of empirical measurements of the analyte to occur. An analyte may be continuously coupled to a solid support for an observable length of time such as, for example, at least about 1 minute, 1 hour (hr), 3 hrs, 6 hrs, 12 hrs, 1 day, 1.5 days, 2 days, 3 days, 1 week (wk), 2 wks, 3 wks, 1 month, or more. The coupling of an analyte to a solid support can occur with a solution-phase chemistry that promotes the deposition of the analyte on the solid support. Coupling of an analyte to a solid support may occur under solution conditions that are optimized for any conceivable solution property, including solution composition, species concentrations, pH, ionic strength, solution temperature, etc. Solution composition can be varied by chemical species, such as buffer type, salts, acids, bases, and surfactants. In some configurations, species such as salts and surfactants may be selected to facilitate the formation of interactions between an analyte and a solid support. Covalent coupling methods for coupling an analyte to a solid support may include species such as catalyst, initiators, and promoters to facilitate particular reactive chemistries.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexmethyldisilazane, alkyl moieties) or hydrophilic moieties (e.g., polyethylene glycol moieties). Surface layers or coatings provided at an interstitial region can comprise linear, branched, or dendrimeric moieties. A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

An analyte-binding site may comprise a plurality of moieties coupled to a solid support. The plurality of moieties can include a coupling moiety and an optional plurality of passivating moieties. Preferably, a moiety containing a coupling moiety may further comprise a passivating moiety. For example, an oligonucleotide coupling moiety may further comprise a PEG passivating moiety. In some configurations, each individual moiety of a plurality of moieties coupled to an analyte-binding site can contain a coupling moiety. Alternatively, in some configurations, only a fraction of moieties of a plurality of moieties coupled to an analyte-binding site may contain a coupling moiety. Coupling moieties and passivating moieties may be provided at an analyte-binding site in a ratio of at least about 1000:1, 100:1, 10:1, 5:1, 2:1, 1:1, 1:2, 1:5, 1:10, 1:100, or 1:1000 coupling-to-passivating moieties. Alternatively or additionally, coupling moieties and passivating moieties may be provided at an analyte-binding site in a ratio of no more than about 1:1000, 1:100, 1:10, 1:5, 1:2, 1:1, 2:1, 5:1, 10:1, 100:1, or 1000:1 coupling-to-passivating moieties.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

An array of analytes may be provided with a characterized or characterizable analyte-binding site occupancy. The analyte-binding site occupancy can be measured as the fraction or percentage of analyte-binding sites of a plurality of analyte-binding sites containing an attached analyte. An array of analytes may be provided with an analyte-binding site occupancy of at least about 10%, 20%, 25%, 30%, 35%, 40%, 45%, 50%, 60%, 70%, 80%, 90%, 95%, 99%, 99.9%, or more than 99.9%. Alternatively or additionally, an array of analytes may be provided with an analyte-binding site occupancy of no more than about 99.9%, 99%, 95%, 90%, 80%, 70%, 60%, 50%, 45%, 40%, 35%, 30%, 25%, 20%, 10%, or less than 10%.

An array of analytes may be provided with a fraction or percentage of individual sites that each contain one and only one analyte. The fraction or percentage may be calculated relative to all other sites in the array including, but not limited to, those containing no analyte and those containing multiple analytes. Preferably, an array of analytes may be provided with super Poisson loading of single analytes (i.e., a fraction or percentage of attachments sites containing one and only one analyte exceeding 37%). An array of analytes may be provided with at least about 10%, 20%, 25%, 30%, 35%, 37%, 40%, 45%, 50%, 60%, 70%, 80%, 90%, 95%, 99%, 99.9%, or more than 99.9% of analyte-binding sites containing one and only one analyte. Alternatively or additionally, an array of analytes may be provided with no more than about 99.9%, 99%, 95%, 90%, 80%, 70%, 60%, 50%, 45%, 40%, 35%, 30%, 25%, 20%, 10%, or less than 10% of analyte-binding sites containing one and only one analyte.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10², 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. Nos. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; or US Pat. App. Pub. No. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g. SNAP) or population thereof has a minimum, maximum or average length of at least about 50 nm, 100 nm, 250 nm, 500 nm, 1 micron, 5 micron or more. Alternatively or additionally, a retaining component (e.g. SNAP) or population thereof has a minimum, maximum or average length of no more than about 5 microns, 1 micron, 500 nm, 250 nm, 100 nm, 50 nm, or less.

Optionally, a retaining component (e.g. SNAP) or population thereof has a minimum, maximum or average volume of at least about 1 micron³, 10 micron³, 100 micron³, 1 mm³ or more. Alternatively or additionally, a retaining component (e.g. SNAP) or population thereof has a minimum, maximum or average volume of no more than about 1 mm³, 100 micron³, 10 micron³, 1 micron³ or less.

Optionally, the minimum, maximum or average area (i.e. footprint) for a retaining component (e.g. SNAP) is at least about 10 nm², 100 nm², 1 micron², 10 micron², 100 micron², 1 mm² or more. Alternatively or additionally, the minimum, maximum or average area for a retaining component (e.g. SNAP) footprint is at most about 1 mm², 100 micron², 10 micron², 1 micron², 100 nm², 10 nm², or less. The footprint of a retaining component (e.g. SNAP) may have a regular shape or an approximately regular shape, such as triangular, square, rectangular, circular, ovoid, or polygonal shape.

A structured nucleic acid particle (e.g. having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

In some configurations, a nucleic acid origami includes a scaffold composed of a nucleic acid strand to which a plurality of oligonucleotides is hybridized. A nucleic acid origami may have a single scaffold molecule or multiple scaffold molecules. A scaffold strand can be linear (i.e. having a 3′ end and 5′ end) or circular (i.e. closed such that the scaffold lacks a 3′ end and 5′ end). A scaffold strand can be derived from a natural source, such as a viral genome or a bacterial plasmid. For example, a nucleic acid scaffold can include a single strand of an M13 viral genome. In other configurations, a scaffold strand may be synthetic, for example, having a non-naturally occurring nucleotide sequence in full or in part. A scaffold nucleic acid can be single stranded but for a plurality of oligonucleotides hybridized thereto or short regions of internal complementarity. The size of a scaffold strand may vary to accommodate different uses. For example, a scaffold strand may include at least about 100, 500, 1000, 2500, 5000 or more nucleotides. Alternatively or additionally, a scaffold strand may include at most about 5000, 2500, 1000, 500, 100 or fewer nucleotides.

A nucleic acid origami can include one or more oligonucleotides that are hybridized to a scaffold strand. An oligonucleotide can include two sequence regions that are hybridized to a scaffold strand, for example, to function as a ‘staple’ that restrains the structure of the scaffold. For example, a single oligonucleotide can hybridize to two regions of a scaffold strand that are separated from each other in the primary sequence of the scaffold strand. As such, the oligonucleotide can function to retain those two regions of the scaffold strand in proximity to each other or to otherwise constrain the scaffold strand to a desired conformation. Two sequence regions of an oligonucleotide staple that bind to a scaffold strand can be adjacent to each other in the nucleotide sequence of the oligonucleotide or separated by a spacer region that does not hybridize to the scaffold strand.

An oligonucleotide can include a first sequence region that is hybridized to a complementary sequence of a nucleic acid origami and a second region that provides a “handle” or “linker” for attaching another moiety. For example, the moiety can include an analyte (e.g. protein), paratope, affinity moiety (e.g. antibody), organic linker, inorganic ion, docker or tether. Optionally, the moiety can be attached to an oligonucleotide that is complementary to the second region of the handle and the moiety can be attached to the nucleic acid origami via hybridization of the handle to the complementary oligonucleotide.

Oligonucleotides can be configured to hybridize with a nucleic acid scaffold, another oligonucleotide, a staple oligonucleotide, or a combination thereof. One or more regions of an oligonucleotide that hybridizes to another sequence of a nucleic acid origami or other structured nucleic acid particle can be located at or near the 5′ end of the oligonucleotide, at or near the 3′ end of the oligonucleotide, or in a region of the oligonucleotide that is between the end regions. The oligonucleotides can be linear (i.e. having a 3′ end and a 5′ end) or closed (i.e. circular, lacking both 3′ and 5′ ends). An oligonucleotide that is included in a nucleic acid origami or other structured nucleic acid particle can have any of a variety of lengths including, for example, at least about 10, 25, 50, 100, 250, 500, or more nucleotides. Alternatively or additionally, an oligonucleotide may have a length of no more than about 500, 250, 100, 50, 25, 10, or fewer nucleotides. An oligonucleotide may form a hybrid of at least about 5, 6, 7, 8, 9, 10, 15, 20, 25, 50 or more consecutive or total base pairs with another nucleotide sequence of a nucleic acid origami. Alternatively or additionally, an oligonucleotide may form a hybrid of no more than about 50, 25, 20, 15, 10, 9, 8, 7, 6, 5, or fewer consecutive or total base pairs with another nucleotide sequence.

A retaining component may be provided with moieties that facilitate a binding interaction with a surface of a solid support, or moieties coupled to the surface of the solid support. Moieties that facilitate coupling of a retaining component to a solid support may be configured to form a covalent interaction or a non-covalent interaction with the solid support or a moiety coupled to the solid support. In an example, a retaining component may be provided with one or more nucleic acid strands that can hybridize to a complementary nucleic acid strand on a surface of a solid support by nucleic acid hybridization. Preferably, a retaining component may be provided with a plurality of moieties that can bind to a surface of a solid support. In some cases, the moieties may be pendant from the retaining component. Pendant moieties may include a linking moiety that increases the length of the moiety and/or increases the flexibility or spatial degrees of freedom of the moiety. A linking moiety can be, for example, a single-stranded nucleic acid (e.g., with a nucleotide sequence that is not complementary to a surface-bound oligonucleotide), a peptide linker, or a synthetic polymer (e.g., polyethylene glycol, alkyl moieties, etc.).

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. Nos. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer. A branched chain polymer can include at least 1, 2, 3, 4, 5, 6, 8 or 10 branch points. Alternatively or additionally, a branched chain can include at most 10, 8, 6, 5, 4, 3, 2 or 1 branch points. A branch point is a covalent intersection between at least two chains. For example, at least 2, 3, 4, 5 or more chains can intersect at a branch point of a branched chain. Alternatively or additionally, at most 5, 4, 3 or 2 chains can intersect at a branch point of a branched chain. A polymer, whether branched or not, can include a single type of monomer subunit or multiple different types of monomer subunits. Accordingly, a polymer can include at least 1, 2, 3, 4, 5 or more different types of monomer subunits. Alternatively or additionally, a polymer can include at most 5, 4, 3, 2 or 1 different types of monomer subunits. A polymer having only one type of subunit in the network of covalent bonds is referred to as a “homopolymer.” In contrast, a “copolymer” includes two or more different types of subunits in the network of covalent bonds.

A retaining component that includes an artificial polymer can have a length, volume or footprint in a range set forth above. A retaining component can be further characterized in terms of molecular weight (or molecular weight distribution) in a desired size range. For example, the molecular weight, average molecular weight distribution, minimum molecular weight distribution or maximum molecular weight distribution can be at least 1 kDa, 2 kDa, 5 kDa, 10 kDa, 25 kDa, 50 kDa or more. Alternatively or additionally, the molecular weight, average molecular weight distribution, minimum molecular weight distribution or maximum molecular weight distribution can be at most 50 kDa, 25 kDa, 10 kDa, 5 kDa, 2 kDa, 1 kDa or less. A retaining component can be characterized in terms of radius of gyration. For example, the radius of gyration can be at least about 2 nm, 5 nm, 10 nm, 15 nm, 25 nm, 50 nm or more. Alternatively or additionally, retaining component can be configured to have a radius of gyration that is at most about 50 nm, 25 nm, 15 nm, 10 nm, 5 nm, 2 nm or less. An artificial polymer can be characterized in term of degree of polymerization (i.e. number of monomer subunits) present. For example, an artificial polymer can include at least 2, 10, 20, 30, 40, 50, 100, 200, 300 or more monomers. Alternatively or additionally, an artificial polymer can include at most 300, 200, 100, 50, 40, 30, 20, 10, or 2 monomers.

An artificial polymer can lack natural polymers or monomers found in natural polymers. For example, the skeletal structure of the artificial polymer can lack natural polymers or monomers. This can be the case whether or not the artificial polymer has attached moieties that include natural polymers or monomers. Examples of natural moieties that can be absent from an artificial polymer, for example in the skeletal structure include, but are not limited to, nucleic acids (e.g. DNA or RNA), nucleotides (e.g. deoxyribonucleotides or ribonucleotides), nucleosides (e.g. deoxyribonucleosides or ribonucleosides), peptides (e.g. proteins, polypeptides or oligopeptides), amino acids, or sugars (e.g. saccharide monomers, monosaccharides, oligosaccharides, polysaccharides or glycans). An artificial polymer can optionally lack any polymer or monomer that is synthesized in vivo or that is capable of being synthesized in vivo. Alternatively, an artificial polymer can include natural moieties that are combined to form a non-naturally occurring molecule. For example, an artificial polymer can be composed of nucleic acid monomers or nucleic acid strands that form a non-naturally occurring nucleic acid dendrimer structure.

Particularly useful artificial polymers include, for example, poly(amidoamine) (PAMAM) dendrimer, poly(amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40: 2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17): 3982 (2020); Zheng, et al. or Chem. Soc. Rev. 44, 4091-4130 (2015), each of which is incorporated herein by reference.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Na⁺, K⁺, Ag⁺, Cu⁺, NH⁴⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti3+, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio)butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl) phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo[2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), α-tocopherol, uric acid, sodium azide, manganese(III)-tetrakis(4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

A fluidic medium may be provided at, heated to, cooled to, or maintained at a temperature of at least about −80 degrees Celsius (° C.), −50° C., −10° C., −5° C., 0° C., 5° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 60° C., 70° C., 80° C., 90° C., 95° C., or more than 95° C. Alternatively or additionally, a fluidic medium may be provided at, heated to, cooled to, or maintained at a temperature of no more than about 95° C., 90° C., 80° C., 70° C., 60° C., 50° C., 45° C., 40° C., 35° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 5° C., 0° C., −5° C., −10° C., −50° C., −80° C., or less than −80° C.

A fluidic medium may be provided at or adjusted to a pH of at least about 0.0, 0.5, 1.0, 1.5, 2.0, 2.5, 3.0, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, 9.0, 9.5, 10.0, 10.5, 11.0, 11.5, 12.0, 12.5, 13.0, 13. 5, 14.0, or more than 14.0. Alternatively or additionally, a fluidic medium may be provided at or adjusted to a pH of no more than about 14.0, 13.5, 13.0, 12.5, 12.0, 11.5, 11.0, 10.5, 10.0, 9.5, 9.0, 8.5, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.5, 5.0, 4.5, 4.0, 3.5, 3.0, 2.5, 2.0, 1.5, 1.0, 0.5, or less than 0.5

A component of a fluidic medium may be provided at or adjusted to a molar concentration of at least about 0.0001 moles per liter (M), 0.001M, 0.01M, 0.02M, 0.03M, 0.04M, 0.05M, 0.06M, 0.07M, 0.08M, 0.09M, 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 7M, 8M, 9M or more than 10M. Alternatively or additionally, a component of a fluidic medium may be provided at or adjusted to a molar concentration of no more than about 10 M, 9M, 8M, 7M, 6M, 5.9M, 5.8M, 5.7M, 5.6M, 5.5M, 5.4M, 5.3M, 5.2M, 5.1M, 5.0M, 4.9M, 4.8M, 4.7M, 4.6M, 4.5M, 4.4M, 4.3M, 4.2M, 4.1M, 4.0M, 3.9M, 3.8M, 3.7M, 3.6M, 3.5M, 3.4M, 3.3M, 3.2M, 3.1M, 3.0M, 2.9M, 2.8M, 2.7M, 2.6M, 2.5M, 2.4M, 2.3M, 2.2M, 2.1M, 2.0M, 1.9M, 1.8M, 1.7M, 1.6M, 1.5M, 1.4M, 1.3M, 1.2M, 1.1M, 1.0M, 0.9M, 0.8M, 0.7M, 0.6M, 0.5M, 0.4M, 0.3M, 0.2M, 0.1M, 0.09M, 0.08M, 0.07M, 0.06M, 0.05M, 0.04M, 0.03M, 0.02M, 0.01M, 0.001M, 0.001M, or less than about 0.001M.

A component of a fluidic medium may be provided at or adjusted to a weight or volumetric percentage of at least about 0.0001%, 0.001%, 0.005%, 0.01%, 0.05%, 0.1%, 0.5%, 1%, 2%, 3%, 4%, 45%, 6%, 7%, 8%, 9%, 10%, 20%, 30%, 40%, 50%, or more than 50%. Alternatively or additionally, a component of a fluidic medium may be provided at or adjusted to a weight or volumetric percentage of no more than about 50%, 40%, 30%, 20%, 10%, 9%, 8%, 7%, 6%, 5%, 4%, 3%, 2%, 1%, 0.5%, 0.1%, 0.05%, 0.01%, 0.005%, 0.001%, 0.0001%, or less than 0.0001%.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants are included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Protoeforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g. sequence agnostic) phosphatase such as alkaline phosphatase can be useful. Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g. histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, α-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb1,3GalNac; and PNGase F can be used to cleave oligosaccharides from asparagines. Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

What is claimed is:

1. A method, comprising:
(a) providing a plurality of polypeptides, wherein each polypeptide of the plurality of polypeptides is individually co-localized with a first unique identifier, and wherein each polypeptide of the plurality of polypeptides has an unknown identity;
(b) contacting the plurality of polypeptides with a plurality of binding reagents, wherein each binding reagent in the plurality of binding reagents comprises a protein substrate or enzymatic cofactor, and wherein the protein substrate or enzymatic cofactor is attached to a second unique identifier,
(c) detecting for each polypeptide of the plurality of polypeptides the presence or absence of a co-localized signal from the second unique identifier and the first unique identifier of the polypeptide; and
(d) for each polypeptide comprising a co-localized signal, assigning protein identity to the polypeptide.

2. The method of claim 1, wherein the protein substrate or enzymatic cofactor is attached to the second unique identifier by a linking moiety.

3. The method of claim 2, wherein the linking moiety comprises a polymer strand.

4. The method of claim 2, wherein the linking moiety comprises a nanoparticle.

5. The method of claim 3 or 4, wherein the linking moiety comprises a polymer nanoparticle, a dendrimer, or a nucleic acid nanoparticle.

6. The method of any one of claims 2-5, wherein the linking moiety is attached to the protein substrate or enzymatic cofactor by a covalent bond between a first functional group of the protein substrate or enzymatic cofactor and a second functional group of the linking moiety.

7. The method of claim 6, wherein the first functional group of the protein substrate or enzymatic cofactor does not bind to a binding site of a target enzyme of the protein substrate or enzymatic cofactor.

8. The method of claims 6 or 7, wherein the protein substrate or enzymatic cofactor comprises a first chiral center.

9. The method of claim 8, wherein the covalent bond between the first functional groups and the second functional group forms a second chiral center.

10. The method of any one of claims 6-9, wherein the second functional group is attached to the linking moiety by a polymer strand comprising one or more hydrophobic residues.

11. The method of any one of claims 6-10, wherein the second functional group is attached to the linking moiety by a polymer strand comprising one or more hydrophilic residues.

12. The method of any one of claims 1-11, wherein the protein substrate or enzymatic cofactor is attached to the second unique identifier by a polymer strand, wherein the polymer strand has an uncoiled region.

13. The method of claim 12, wherein the uncoiled region is uncoiled in the same fluidic medium as the target enzyme of the binding reagent is active.

14. The method of any one of claims 1-13, wherein the first unique identifier comprises an address of a solid support.

15. The method of claim 14, wherein the second unique identifier comprises a fluorophore or luminophore.

16. The method of claim 15, wherein detecting for each polypeptide of the plurality of polypeptides the presence or absence of the co-localized signal from the second unique identifier with the first unique identifier of the polypeptide comprises detecting an optical signal from the fluorophore or luminophore at the address of the solid support.

17. The method of any one of claims 14-16, wherein the solid support comprises a plurality of addresses, each address being separated by an optically resolvable distance from any other address of the plurality of addresses.

18. The method of claim 17, wherein the plurality of polypeptides is immobilized on the solid support, wherein each address is attached to only one polypeptide of the plurality of polypeptides.

19. The method of claim 18, wherein each polypeptide of the plurality of polypeptides is attached to only one particle, wherein the particle is immobilized to an address of the plurality of addresses.

20. The method of any one of claims 14-19, further comprising contacting to the plurality of polypeptides a plurality of second binding reagents, wherein each second binding reagent comprises a second protein substrate or enzymatic cofactor, and wherein the second protein substrate or enzymatic cofactor is attached to a third unique identifier, wherein the third unique identifier comprises a fluorophore or a luminophore.

21. The method of claim 20, wherein detecting for each polypeptide of the plurality of polypeptides presence or absence of a co-localized signal from the second unique identifier with the first unique identifier of the polypeptide further comprises detecting for each polypeptide of the plurality of polypeptides presence or absence of a co-localized optical signal from the third unique identifier with the first unique identifier of the polypeptide.

22. The method of claim 21, wherein, for a first polypeptide of the plurality of polypeptides, the presence of an optical signal from the second unique identifier and the presence of an optical signal from the third unique identifier is detected.

23. The method of claim 22, wherein the first polypeptide of the plurality of polypeptides is assigned a first enzyme identity.

24. The method of claim 23, wherein, for a second polypeptide of the plurality of polypeptides, the presence of an optical signal from the second unique identifier and the absence of an optical signal from the third unique identifier is detected.

25. The method of claim 24, wherein the second polypeptide of the plurality of polypeptides is assigned a second enzyme identity, wherein the first enzyme identity differs from the second enzyme identity.

26. The method of any one of claims 1-13, wherein the first unique identifier comprises a first barcode moiety.

27. The method of claim 26, wherein the second unique identifier comprises a second barcode moiety.

28. The method of claim 27, wherein detecting for each polypeptide of the plurality of polypeptides the presence or absence of the co-localized signal from the second unique identifier with the first unique identifier of the polypeptide comprises detecting an interaction identification moiety comprising the first barcode moiety and the second barcode moiety.

29. The method of claim 28, further comprising forming the interaction identification moiety.

30. The method of claim 29, wherein forming the interaction identification moiety comprises ligating the first barcode moiety to the second barcode moiety.

31. The method of claims 29 or 30, wherein the first barcode moiety or the second barcode moiety comprises a peptide barcode moiety.

32. The method of claims 29 or 30, wherein the first barcode moiety or the second barcode moiety comprises a nucleic acid barcode moiety.

33. The method of claim 32, wherein forming the interaction identification moiety comprises extending the first barcode moiety or the second barcode moiety by a polymerase extension reaction, wherein the polymerase extension reaction forms a complementary sequence of one of the barcode moieties.

34. The method of any one of claims 26-33, further comprising contacting to the plurality of polypeptides a plurality of second binding reagents, wherein each second binding reagent comprises a second protein substrate or enzymatic cofactor, and wherein the second protein substrate or enzymatic cofactor is attached to a third unique identifier, wherein the third unique identifier comprises a third barcode moiety.

35. The method of claim 34, wherein the second barcode moiety is complementary to the third barcode moiety.

36. The method of claim 35, further comprising coupling the second barcode moiety to the third barcode moiety.

37. The method of claim 36, further comprising coupling the protein substrate or enzymatic cofactor to the second protein substrate or enzymatic cofactor.

38. The method of claim 37, further comprising, after coupling the protein substrate or enzymatic cofactor to the second protein substrate or enzymatic cofactor, forming an interaction identification moiety comprising the first unique identifier and the third unique identifier.

39. The method of claim 38, wherein detecting for each polypeptide of the plurality of polypeptides presence or absence of the co-localized signal from the second unique identifier with the first unique identifier of the polypeptide comprises detecting the interaction identification moiety.

40. The method of any one of claims 26-39, wherein providing the plurality of polypeptides comprises providing the plurality of polypeptides immobilized on a solid support.

41. The method of claim 40, wherein contacting the plurality of polypeptides with the plurality of binding reagents comprises contacting the plurality of binding reagents with the solid support.

42. The method of any one of claims 26-39, wherein providing the plurality of polypeptides comprises providing the plurality of polypeptides in a fluid phase.

43. The method of claim 42, wherein contacting the plurality of polypeptides with the plurality of binding reagents comprises contacting the plurality of binding reagents with the plurality of polypeptides in the fluid phase.

44. A method, comprising:
(a) providing a library of binding reagents, wherein each binding reagent of the library of binding reagents comprises a protein substrate or enzymatic cofactor attached to a first unique identifier, wherein the library of binding reagents comprises a plurality of structurally-unique binding reagents, each structurally unique binding reagent comprising a differing attachment conformation of the protein substrate or enzymatic cofactor to the first unique identifier, and each structurally unique binding reagent comprising a first unique identifier that differs from the first unique identifier of any other structurally-unique binding reagent;
(b) contacting a protein molecule with the library of binding reagents, wherein the protein molecule is co-localized with a second unique identifier; and
(c) detecting co-localization of a first unique identifier of a structurally unique binding reagent with the second unique identifier, thereby identifying a portion of the protein substrate or enzymatic cofactor that is bound by the protein molecule.

45. The method of claim 44, wherein the plurality of structurally unique binding reagents further comprises a derivative of the protein substrate or enzymatic cofactor.

46. The method of claim 45, wherein the derivative of the protein substrate or enzymatic cofactor comprises an addition of a functional group, a subtraction of a functional group, a substitution of a functional group, or a combination thereof.

47. A method, comprising:
(a) providing a library of binding reagents, wherein each binding reagent of the library of binding reagents comprises protein binding candidate attached to a first unique identifier, wherein the library of binding reagents comprises a plurality of structurally-unique binding reagents, each structurally unique binding reagent comprising a differing enzyme binding candidate, and each structurally unique binding reagent comprising a first unique identifier that differs from the first unique identifier of any other structurally-unique binding reagent;
(b) contacting a protein molecule with the library of binding reagents; wherein the protein molecule is co-localized with a second unique identifier; and
(c) detecting co-localization of a first unique identifier of a structurally unique binding reagent with the second unique identifier, thereby identifying a molecule that binds to the protein molecule.

48. The method of claim 47, wherein the protein binding candidate comprises a protein substrate.

49. The method of claim 47, wherein the protein binding candidate comprises an enzymatic cofactor.

50. The method of claim 47, wherein the protein binding candidate comprises a pharmaceutical compound.

51. The method of claim 47, wherein the protein binding candidate comprises a toxin.

52. A method, comprising:
(a) contacting a protein molecule to a binding reagent in the presence of a molecular activator, wherein the protein molecule is co-localized with a first unique identifier, wherein the binding reagent comprises a protein substrate, and wherein the protein substrate is attached to a second unique identifier; and
(b) detecting a co-localized signal from the second unique identifier of the binding reagent and the first unique identifier of the polypeptide, thereby identifying binding of the protein substrate to the protein molecule.

53. The method of claim 52, further comprising attaching the molecular activator to the protein substrate.

54. The method of claim 53, further comprising, after attaching the molecular activator to the protein substrate, binding the binding reagent to the protein molecule.

55. The method of claim 53, wherein attaching the molecular activator to the protein substrate comprises: i) binding the molecular activator to the protein molecule, ii) binding the protein substrate to the protein molecule, and iii) attaching the molecular activator to the protein substrate by the activity of the protein molecule.

56. A method, comprising:
(a) providing a plurality of polypeptides, wherein the plurality of polypeptides is immobilized on a plurality of sites of a solid support, wherein each site of the plurality of sites is attached to only one polypeptide of the plurality of polypeptides, and wherein each site of the plurality of sites is optically resolvable from each other site of the plurality of sites;
(b) contacting the solid support with a plurality of binding reagents, wherein each binding reagent in the plurality of binding reagents comprises a protein substrate or enzymatic cofactor, and wherein the protein substrate or enzymatic cofactor is attached to a detectable label,
(c) detecting for each site of the plurality of sites a presence or absence of a signal from a detectable label; and
(d) for each site of the plurality of sites having a detected presence of a signal from the detectable label, assigning a protein identity to the polypeptide of the plurality of polypeptides attached to the site.

57. A method, comprising:
(a) combining in a fluid phase a plurality of polypeptides with a plurality of binding reagents, wherein each polypeptide of the plurality of polypeptides is individually attached to a first unique identifier and has an unknown identity, wherein each binding reagent in the plurality of binding reagents comprises a protein substrate or enzymatic cofactor, and wherein the protein substrate or enzymatic cofactor of each individual binding reagent is attached to a second unique identifier;
(b) coupling binding reagents of the plurality of binding reagents to polypeptides of the plurality of polypeptides;
(c) for each polypeptide of the polypeptides bound to a binding reagent of the plurality of binding reagents, forming an interaction identification moiety, wherein the interaction identification moiety comprises the first unique identifier of the polypeptide and the second unique identifier of the binding reagent;
(d) detecting each formed interaction identification moiety; and
(e) for each detected interaction identification moiety, determining an identity of the polypeptide from which the interaction identification moiety was obtained.
